(19) United States
(12) Patent Application Publication
(10) Pub. No.: US 2011/0207801 A1
(43) Pub. Date: Aug. 25, 2011

(54) Novel Genes, Compositions, and Methods for Modulating the Unfolded Protein Response

(75) Inventors: Randal J. Kaufman, Ann Arbor, MI (US); Kyungo Lee, Seoul (KR); Kazutoshi Mori, Kyoto (JP); Hiderou Yoshida, Ritto (JP)

(73) Assignee: University of Michigan, Ann Arbor, MI (US)

(21) Appl. No.: 12/649,613

(22) Filed: Dec. 30, 2009

Related U.S. Application Data
(63) Continuation of application No. 10/971,994, filed on Oct. 21, 2004, now abandoned.
Application No. 10/971,994 is a continuation of application No. PCT/US03/12640, filed on Apr. 22, 2003.
(60) Provisional application No. 60/375,098, filed on Apr. 22, 2002.
(60) Provisional application No. 60/374,880, filed on Apr. 23, 2002.

(51) Int. Cl. A61K 31/7088; C12N 5/10; C12P 21/00; C12N 1/00; A61P 37/04; A61P 43/00; A61P 11/00

(52) U.S. Cl. 514/44 R; 435/325; 435/69.1; 435/243

(57) ABSTRACT

The present invention relates to methods and compositions for modulating the unfolded protein response. The method further relates to methods and compositions for the treatment and diagnosis of protein conformational diseases or disorders, including, but not limited to, α1-antitrypsin deficiency, cystic fibrosis, and autoimmune diseases and disorders. The invention further provides methods for modulating the unfolded protein response by modulating XBP1 mRNA splicing.

### RELATED APPLICATIONS

The present application is a Continuation application of PCT Application No. PCT/US2003/012640 filed on Apr. 22, 2003, which claims priority to U.S. Provisional Patent Application Ser. No. 60/375,098, filed Apr. 22, 2002 and U.S. Provisional Patent Application Ser. No. 60/374,880, filed Apr. 23, 2002, all hereby expressly incorporated by reference.

### BACKGROUND OF THE INVENTION

Protein conformational diseases or disorders, such as α1-antitrypsin deficiency and cystic fibrosis, are associated with the accumulation of unfolded proteins in the endoplasmic reticulum (also referred to as “ER”) (Aridor et al., 1999; Kaufman, 1999; Kopito et al., 2000). Expression of mutant or even some wild-type proteins, viral infection, energy or nutrient depletion, extreme environmental conditions, or stimuli that elicit excessive calcium release from the ER lumen compromise protein-folding reactions in the ER, causing unfolded proteins to accumulate, and initiate signals that are transmitted to the cytoplasm and nucleus. This adaptive response includes: 1) the transcriptional activation of genes encoding ER-resident chaperones and folding catalysts and protein degrading complexes that augment ER folding capacity, and 2) translational attenuation to limit further accumulation of unfolded proteins in the ER (Kaufman, 1999; Mori, 2000). In mammals, this signal transduction cascade, termed the unfolded protein response (also referred to herein as “UPR”), is mediated by three types of ER transmembrane proteins: the protein-kinase and site-specific endoribonuclease IRE1 (Tirasophon et al., 1998; Wang et al., 1998); the eukaryotic translation initiation factor 2 kinase, PERK/PEK (Shi et al., 1998; Harding et al., 1999); and the transcriptional activator ATF6 (Yoshida et al., 1998 and 2001a). If adaptation is not sufficient, an apoptotic response is initiated leading to activation of JNK protein kinase and caspases 7, 12, and 3 (Urano et al., 2000a; Nakagawa et al., 2000; Yoneda et al., 2001).

In Saccharomyces cerevisiae, the UPR is controlled by the ER transmembrane protein kinase/endoribonuclease IRE1p (Nikawa et al., 1992; Cox et al., 1993; Mori et al., 1993). Following ER stress, IRE1p is essential for survival by initiating splicing of the mRNA encoding the basic-leucine zipper (bZIP) transcription factor Hac1p (Chapman and Walter, 1997; Kawahara et al., 1997; Mori et al., 2000). Whereas unspliced HAC1 mRNA is poorly translated, spliced HAC1 mRNA is efficiently translated to yield a protein that acts as a more potent transcriptional activator (Chapman and Walter, 1997; Kawahara et al., 1997; Mori et al., 2000). As the cellular level of Hac1p increases, the transcription of genes harboring UPR elements (also referred to herein as “UPREs”) in their promoters is activated.

In the mammalian genome, there are two homologues of yeast IRE1, IRE1α and IRE1β. Whereas IRE1α is expressed in all cells and tissues, IRE1β expression is primarily restricted to intestinal epithelial cells (Bertolotti et al., 2000). Upon over-expression, the endoribonuclease of either IRE1α or IRE β is sufficient to activate the UPR transcriptional response (Tirasophon et al., 1998 and 2000; Wang et al., 1998). Therefore, IRE1-mediated splicing of an RNA target is likely one mechanism that activates the UPR. However, a HAC1 homologue has not been identified in the sequenced genomes of C. elegans or D. melanogaster, or in the sequences available from the human or murine genomes. Interestingly, deletion of either or both the IRE1α and IRE1β genes did not interfere with transcriptional activation of several UPR genes or survival following ER stress in cultured mouse cells (Urano et al., 2000a and 2000b; Kaufman et al., 2001). Therefore, at least one additional inductive and adaptive mechanism exists. Finally, over-expression of either IRE1α or IRE1β was also linked to apoptosis, leading to the question as to whether these pathways are adaptive or apoptotic responses to ER stress (Iwawaki et al., 2001).

### SUMMARY OF THE INVENTION

The present invention provides methods and compositions for the diagnosis and treatment of protein conformational diseases or disorders. The present invention is based, at least in part, on the discovery that IRE1α splices XBP1 (X-box binding potential) mRNA to generate a new C terminus, thereby converting it into an unfolded protein response (UPR) transcriptional activator. In particular, IRE1α removes a nonconventional 26-nucleotide intron which results in a spliced form of XBP1 with increased transactivation potential. In addition, it has been found that ATF6 increases the amount of XBP1 mRNA. It has thus been found that both processing of ATF6 and IRE1α-mediated splicing of XBP1 mRNA are required for full activation of the UPR. Thus, spliced XBP1 mRNA can activate the UPR to treat protein conformational diseases and disorders. Diagnostic targets and therapeutic agents to enhance protein folding capabilities and limit the folding load on the ER are therefore provided.

In one embodiment, the invention features an isolated nucleic acid molecule that includes the nucleotide sequence set forth in SEQ ID NO:1 or SEQ ID NO:3, wherein SEQ ID NO:1 is the spliced XBP1 mRNA sequence and SEQ ID NO:3 is the cDNA sequence derived from the spliced XBP1 mRNA sequence. In another embodiment, the invention features an isolated nucleic acid molecule that encodes a polypeptide including the amino acid sequence set forth in SEQ ID NO:2, wherein SEQ ID NO:2 is the amino acid sequence of spliced XBP1.

In still other embodiments, the invention features isolated nucleic acid molecules including nucleotide sequences that are substantially identical (e.g., 60%, 63%, 65%, 67%, 69%, 71.2%, 72%, 75%, 80%, 85%, 90%, 95%, 96%, 97%, 98% or 99% identical) to the nucleotide sequence set forth as SEQ ID NO:1 or SEQ ID NO:3. In another embodiment, the invention features isolated nucleic acid molecules which encode a polypeptide including an amino acid sequence that is substantially identical (60%, 63%, 65%, 67%, 69%, 71.2%, 72%, 75%, 80%, 85%, 90%, 95%, 96%, 97%, 98% or 99% identical) to the amino acid sequence set forth as SEQ ID NO:2. In still other embodiments, the invention features nucleic acid molecules that are complementary to, antisense to, or hybridize under stringent conditions to the isolated nucleic acid molecules described herein.

In another aspect, the invention provides vectors including the isolated nucleic acid molecules described herein. Such vectors can optionally include nucleotide sequences encoding heterologous polypeptides. Also featured are host cells including such vectors (e.g., host cells including vectors suitable for producing nucleic acid molecules and polypeptides of the present invention).

Another embodiment features a polypeptide including the amino acid sequence set forth as SEQ ID NO:2, a polypeptide including an amino acid sequence at least 60%, 63%, 65%, 67%, 69%, 71.2%, 72%, 75%, 80%, 85%, 90%, 95%, 96%, 97%, 98% or 99% identical to the amino acid sequence set forth as SEQ ID NO:2, a polypeptide encoded by a nucleic acid molecule including a nucleotide sequence at least 60%, 63%, 65%, 67%, 69%, 71.2%, 72%, 75%, 80%, 85%, 90%, 95%, 96%, 97%, 98% or 99% identical to the nucleotide sequence set forth as SEQ ID NO:1 or SEQ ID NO:3.

In a related aspect, the invention features antibodies (e.g., antibodies which specifically bind to any one of the polypeptides described herein) as well as fusion polypeptides including all or a fragment of a polypeptide described herein.

The present invention further features methods for detecting XBP1 polypeptides and/or spliced XBP1 nucleic acid molecules, such methods featuring, for example, a probe, primer or antibody described herein. Also featured are kits e.g., kits for the detection of XBP1 polypeptides and/or spliced XBP1 nucleic acid molecules.

In a related aspect, the invention features methods for identifying compounds which bind to and/or modulate the activity of an XBP1 polypeptide or spliced XBP1 mRNA molecule described herein. The method further includes contacting a XBP1 polypeptide with a test compound and determining the effect of the test compound on the activity of the polypeptide. In another embodiment, the invention features a compound, wherein the ability of the compound to modulate the production of spliced XBP1 mRNA or spliced XBP1 polypeptide activity is determined by detecting accumulation of unfolded proteins in the endoplasmic reticulum. In still another embodiment, the invention provides a compound, wherein the ability of the compound to modulate the production of spliced XBP1 mRNA or spliced XBP1 polypeptide activity is determined by detecting spliced XBP1 mRNA levels.

In one aspect, the invention provides methods for identifying a compound capable of treating a protein conformational disease or disorder, e.g., cystic fibrosis, α1-antitrypsin deficiency and/or autoimmune diseases and disorders. The method includes identifying a compound that modulates the production of spliced XBP1 mRNA and spliced XBP1 polypeptide activity.

In yet another embodiment, the present invention provides a method for increasing XBP1 transactivation potential, comprising inducing IRE1 to splice an mRNA molecule that encodes XBP1. In still another embodiment, the mRNA molecule is spliced by removal of a 26-nucleotide intron.

In another embodiment, the present invention provides a method for activating the unfolded protein response, comprising splicing XBP1 mRNA by IRE1 and one or more of the following steps: site 2 protease mediated cleavage of ATF6 and PEK phosphorylation of the a subunit of eukaryotic translation initiation factor 2 (e1F2α) at Ser⁵¹.

In another embodiment, the present invention provides a method for identifying a compound capable of modulating a protein conformational disease or disorder comprising contacting a cell which is capable of producing spliced XBP1 mRNA with a test compound and assaying the ability of the test compound to modulate the production of spliced XBP1 mRNA or the activity of a spliced XBP1 polypeptide, thereby identifying a compound capable of modulating a protein conformational disease or disorder.

In another embodiment, the present invention provides a method for modulating the unfolded protein response in a cell comprising contacting a cell with a modulator of XBP1 mRNA splicing, thereby modulating the unfolded protein response. In yet another embodiment, the cell is an epithelial cell. In still another embodiment, the XBP1 modulator is a small molecule. In another embodiment, the invention features a XBP1 modulator, which is capable of modulating spliced XBP1 polypeptide activity or spliced XBP1 nucleic acid expression.

In another embodiment, the present invention provides a method of detecting IRE1 activation in a sample comprising PCR analysis of XBP1 RNA. In yet another embodiment, the PCR primers amplify the region encompassing the overlap between open reading frame 1 (ORF1) and open reading frame 2 (ORF2) within the XBP1 mRNA.

In another embodiment, the present invention provides a construct which contains XBP1 mRNA and a gene of interest, wherein the coding region of the gene of interest is downstream from the XBP1 intron, wherein the XBP1 intron may be spliced by activation of IRE1. The invention further provides a virus, cell or nonhuman animal carrying the construct described above.

In another embodiment, the present invention provides a method of detecting IRE1 activation in a sample comprising monitoring the expression of a reporter gene that is regulated by splicing of the XBP1 intron. In still another embodiment, the invention features detection of IRE1 activation, wherein the reporter gene is fused to the XBP1 open reading frame 1 (ORF1) downstream of the XBP1 intron.

In another embodiment, the invention features a method for inhibiting the XBP1 pathway by blocking IRE 1 activation.

In another embodiment, the present invention provides a method for treating a subject with a protein conformational disease or disorder comprising inducing IRE1 to splice an mRNA molecule that encodes XBP1, thereby activating the unfolded protein response. In yet another embodiment, the invention features inducing ATF6 mediated production of XBP1 mRNA.

In another embodiment, the present invention provides a method for treating an autoimmune disease or disorder by decreasing the unfolded protein response by inhibiting XBP1 transactivation, thereby decreasing the differentiation of B cells to plasma cells, to treat an autoimmune disease or disorder. The invention further provides treatment of an autoimmune disease or disorder, wherein the autoimmune disease or disorder is selected from the group consisting of multiple sclerosis, muscular dystrophy, lupus, and arthritis.

In another embodiment, the present invention provides a nonhuman transgenic animal carrying a transfected DNA molecule or transgene, wherein the transfected DNA molecule or transgene contains elements of the XBP1 intron that are essential for functional splicing of the XBP1 mRNA molecule. In yet another embodiment, the invention features a nonhuman transgenic animal carrying a transgene encoding the spliced XBP1 mRNA. In still another embodiment, the invention provides a nonhuman homologous recombinant animal which contains cells that have an altered XBP1 gene.

### BRIEF DESCRIPTION OF THE DRAWINGS

FIG. 1 depicts the human spliced XBP1 cDNA to mRNA sequence. The nucleotide sequence corresponds to nucleic acids 1-1761 of SEQ ID NO:1.

FIG. 2 depicts the predicted amino acid sequence of the XBP1 polypeptide. The amino acid sequence corresponds to amino acids 1-376 of SEQ ID NO:2.

FIG. 3 depicts the cDNA sequence derived from the human spliced XBP1 mRNA. The cDNA sequence corresponds to nucleic acids 1-1761 of SEQ ID NO:3.

FIG. 4 depicts the cDNA sequence of human unspliced XBP1. The nucleic acid sequence corresponds to nucleic acids 1-1787 of SEQ ID NO:4.

FIGS. 5A-5C demonstrate that C. elegans has an unfolded protein response.

FIG. 5A depicts a Northern blot analysis of hsp-3 expression in response to DTT.

FIG. 5B depicts a quantitative Taqman RT-PCR analysis of hsp-3 and hsp-4 expression. Expression of hsp-3 and hsp-4 was normalized to act-1/act-3. The error bars represent standard deviation calculated from three reactions.

FIG. 5C depicts potential UPR regulatory elements in the promoters of hsp-3 and hsp-4. Numbers are relative to the ATG start codon. The asterisk symbol indicates the sequence marked is the complementary sequence.

FIGS. 6A-6E demonstrate IRE1 and PEK1 signal redundant pathways required for larval development.

FIG. 6A depicts isolation of IRE1(v33) and PEK1(ok275) deletion alleles. The IRE1 gene (C41C4.4) maps to Chromosome II, and features one unusually large intron (˜7.7 kb, indicated by - - - ) that contains a second gene: unc-105. The PEK1 gene (F46C3.1) maps to Chromosome X. The positions of primers used for PCR reactions are depicted, regions deleted are indicated, and the transmembrane domain is indicated by TM.

FIG. 6B depicts isolation of IRE1(v33); PEK1(ok275) homozygotes. IRE1(v33)/mnC1; PEK1(ok275) animals are wild-type and mnC1; PEK1(ok275) are Dpy Uncs.

FIG. 6C depicts PCR analysis which confirmed that L2-arrested animals were IRE1(v33); PEK1(ok275) homozygotes. Lane 1, N2; Lane 2, IRE1(v33); Lane 3, PEK1(ok275); Lane 4, IRE1(v33)/mnC1, PEK1(ok275); and Lanes 5-7 are L2-arrested larvae. The internal control reaction using primers PF2/PR5 yielded a 320 bp band. (i) To analyze the IRE1 gene, primers from inside the IRE1 deletion (T7IF/T7IR) were used. IRE1(v33) (lane 2) and L2-arrested larvae (lanes 5-7) were missing a 680 bp band. (ii) To analyze the PEK1 gene, the primers PF5 from inside the PEK1 deletion and PR2 were used. All of PEK1(ok275) homozygotes and L2-arrested larvae were missing a 630 bp band (lanes 3-7).

FIGS. 6D and 6E depict Nomarski micrographs of a 3-day old IRE1(v33); PEK1(ok275) mutant (Figure D) and IRE1(RNAi); PEK1(RNAi) animal (Figure E). Open arrows indicate vacuoles in intestinal cells. Closed arrows indicate the nuclei of necrotic intestinal cells. The germ line IRE1(v33); PEK1(ok275) animals did not develop past the L2 stage.

FIGS. 7A-7F demonstrate that C. elegans xbp-1 mRNA is the substrate for the endoribonuclease activity of IRE1.

FIG. 7A depicts a schematic representation of the two large open reading frames encoded by xbp-1 transcripts before and after stress-induced splicing. Numbers above the mRNA denote nucleotide positions with the translation start site set at 1. Following ER stress, splicing of an unconventional intron between nucleotides 451 and 475 results in a combined, longer ORF. Primers T7-R743F/R743-3R (arrows) were used to detect xbp-1 spliced products and to prepare templates for in vitro cleavage.

FIG. 7B depicts the nucleotide sequences from cDNAs corresponding to unspliced and spliced forms of xbp-1 mRNA.

FIG. 7C depicts that time-course of xbp-1 mRNA splicing. RNA prepared from drug-treated wild-type (N2) L2 larvae and mixed-staged IRE1(v33) mutants, was analyzed by RT-PCR and agarose gel electrophoresis.

FIG. 7D depicts the secondary structures of the splice sites of C. elegans xbp-1 mRNAs and S. cerevisiae HAC1. The six nucleotides that are indispensable for the yeast HAC1 mRNA cleavage reaction, are conserved in xbp-1 mRNA and underlined in the diagram. Based on the HAC1 mRNA cleavage reaction, the potential cleavage sites in xbp-1 mRNA were identified, as indicated by the arrowheads. Cleavage at these two sites and subsequent ligation of the 5′ and 3′ fragments would yield the spliced form of xbp-1 mRNA. FIG. 7E depicts a Western blot analysis of human IRE1α wild-type and endoribonuclease mutant (K907A) expressed in COS-1 cells.

FIG. 7F depicts in vitro cleavage of C. elegans xbp-1 RNA by human IRE1α. Human IRE1α protein was immunoprecipitated and incubated with the xbp-1 RNA substrates (399 nt). Wild-type xbp-1 RNA yields two cleavage fragments (266 nt and 110 nt) detected by polyacrylamide gel electrophoresis (lane 3). Each mutant prepared was a transition mutation. Cleavage in the 5′ and 3′ loops were detected by the appearance of 289 nt (lanes 5, 9, 11) and 133 nt (lanes 14, 16 and 17) fragments, respectively. Control reactions were performed without adding immunoprecipitated hIRE1α proteins (lanes 1, 4, 6, 8 and 10).

FIGS. 8A-8B demonstrate that RNAi shows that C. elegans xbp-1 and PEK1 are redundant genes required for larval development.

FIG. 8A depicts growth of PEK1(ok275); xbp-1(RNAi). Though PEK1(ok275); xbp-1(RNAi) eggs hatched normally, significant death (51% of 723 hatched larvae) was observed at day 2 after eggs were laid. By 5 days, nearly 98% of PEK1(ok275); xbp-1(RNAi) larvae were dead.

FIG. 8B depicts a Nomarski micrograph of a 2.5-day old PEK1(ok275); xbp-1(RNAi) L2 larvae. The worm is oriented with anterior to the right and ventral up. Open arrows indicate vacuoles present in the intestinal cells. Many distinct granules are also observed.

FIGS. 9A-9B demonstrate that IRE1 and xbp-1 are required for the UPR in C. elegans. Individual 1.5 day-old L2 larvae were treated with M9 buffer (control), DTT (2.5 mM) or tunicamycin (28 μg/ml) for 4 hours. Each column in the figure represents one independent treatment and RNA isolation. Expression of hsp-3 and hsp-4 was normalized to that of act-1/act-3. The error bars show standard deviation based on the normalized duplicate or triplicate reactions. Standard deviations (SD) were calculated from independent experiments.

FIG. 9A depicts the relative expression of hsp-3.

FIG. 9B depicts the relative expression of hsp-4.

FIGS. 10A-10C demonstrate that mutant animals are more sensitive to tunicamycin. Eggs from each strain were laid on plates containing different concentrations of tunicamycin, counted, and studied after 3 days. The number of eggs studied is listed above each column. The X-axis represents tunicamycin concentration. The total worm population was grouped into three fractions: animals that matured to L4 or older, animals that arrested at or prior to the L3 stage, and dead animals. Each fraction was plotted as the percentage of total eggs laid (Y-axis).

FIG. 10A represents N2 animals.

FIG. 10B represents IRE1(v33) mutants.

FIG. 10C represents PEK1(ok275) mutants.

FIGS. 11A-11B demonstrate which pathways signal the UPR during development in C. elegans and S. cerevisiae. Pathways that are supported by directed evidence are printed in bold.

FIG. 11A depicts the UPR in C. elegans.

FIG. 11B depicts the UPR in S. cerevisiae.

FIGS. 12A-12G demonstrate the generation and characterization of IRE1α-null MEFs.

FIG. 12A depicts a schematic representation of the predicted recombination of targeting vector and the mIRE1α locus. The bar indicates position of a 0.5-kb BamHI-XhoI fragment used as probe for Southern hybridization.

FIG. 12B depicts a Southern analysis of ES recombinant clones (1A9 and 1H10) compared to the parental R1 cells.

FIG. 12C depicts a Northern blot analysis of IRE1α-null MEFs. Wild-type and IRE1α-null MEFs were treated with or without 10 μg/ml tunicamycin for 6 hr prior to harvesting total RNA for Northern blot analysis. The blot was probed with [α-³²P]-labeled 3.6-kb EcoRI-XbaI fragment from pED-hIRE1α cDNA.

FIG. 12D depicts a Western blot analysis of wild-type and IRE1α-null MEFs. Proteins were prepared from wild-type and IRE1α-null MEFs (lanes 1 and 2) and from the pancreatic β-cell line HIT-T15 (lane 3). Phosphorylated and nonphosphorylated forms of IRE1α are indicated.

FIGS. 12E and 12F depict a Northern blot analysis of wild-type and IRE1α-null MEFs. One blot was probed with [α-³²P]-labeled hamster BiP cDNA and β-actin cDNA (E) and another blot was probed with [α-³²P]-labeled mouse GRP94 DNA and β-actin cDNA (F). Quantification of the results, which showed that tunicamycin induced GRP94 mRNA 6.7- and 5.4-fold in wild-type and IRE1α-null MEFs, respectively.

FIG. 12G depicts BiP reporter gene expression in IRE1α-null MEFs. The reporter plasmids containing the luciferase gene under control of rat BiP promoter and β-galactosidase under control of the CMV promoter were cotransfected into wild-type and IRE1α-null MEFs. The transfected cells were treated with 2 μg/ml tunicamycin for 16 hr prior to harvest. The luciferase activities are presented relative to CMV β-galactosidase activities.

FIGS. 13A-13D demonstrate 5× ATF6 reporter activation is defective in IRE1α-null MEFs.

FIG. 13A depicts 5× ATF6 reporter gene expression in wild-type and IRE1α-null MEFs. The reporter plasmids containing the luciferase gene under control of 5× ATF6 binding sites and β-galactosidase under control of the CMV promoter were cotransfected into wild-type and IRE1α-null MEFs. The transfected cells were treated with 2 μg/ml tunicamycin for 16 hours prior to harvest. The luciferase activities are presented relative to CMV β-galactosidase activities.

FIGS. 13B, 13C and 13D depict wild-type and IRE1α-null MEFs that were transfected as above in the presence of vector alone or vector containing wild-type IRE1α, kinase-defective (K599A) IRE1α, RNase defective (K907A) IRE1α, C-terminal deleted IRE1α (IRE1α ΔC), ATF2, ATF4, ATF6, processed form of ATF6 (ATF6 50-kDa), c-Jun, or c-Fos as indicated. The vector used for IRE1α expression was pEDΔC. The empty vectors used as controls were pEDΔC (B and D), pcDNA3 (Vector 1) and pCMV-HA (Vector 2) (C). MEFs were transfected by either Effectine (B and D) or FuGENE6 (C) according to the manufacture's recommended procedures. The transfected cells were treated with 10 μg/ml tunicamycin for 6 hours (B and D) or 2 μg/ml tunicamycin for 16 hours (C) prior to harvest.

FIGS. 14A-14C demonstrate that IRE1α is not required for ATF6 cleavage, nuclear translocation, or transcriptional activation.

FIG. 14A depicts a Western blot analysis of ATF6. Wild-type and IRE1α-null MEFs were treated with tunicamycin (10 μg/ml) for increasing times and protein extracts were prepared for Western blot analysis. ATF6 proteins were detected using anti-ATF6 antibody and anti-rabbit immunoglobulin conjugated with horseradish peroxidase and enhanced chemiluminescence.

FIG. 14B depicts a Pulse-chase analysis of ATF6. Wild-type and IRE1α-null MEFs were pulse-labeled with [³⁵S]-methionine and [³⁵S]-cysteine (0.5 mCi/100-mm dish) for 40 minutes and then chase was performed with or without 10 μg/ml tunicamycin for the periods indicated. Proteins were extracted and immunoprecipitated using anti-ATF6 antibody. Immunoprecipitates were subjected to SDS-PAGE and radiolabeled proteins were visualized using PhosphoImager (Molecular Dynamics).

FIG. 14C depicts ATF6 cleavage-dependent GAL4 reporter gene expression in wild-type and IRE1α-null MEFs. The reporter plasmids containing the luciferase gene under control of GAL4 promoter and β-galactosidase under control of the CMV promoter were cotransfected with the GAL4 DNA binding domain-ATF6 fusion protein expression vector into wild-type and IRE1α-null MEFs. The transfected cells were treated with 2 μg/ml tunicamycin for 16 hours prior to harvest. The luciferase activities are presented relative to CMV β-galactosidase activities. The diagram on the left depicts ATF6 cleavage-dependent GAL4 reporter gene expression that is independent from the transcriptional activity of endogenous ATF6.

FIGS. 15A-15H demonstrate that 5× ATF6 reporter induction requires IRE1α-dependent splicing of XBP1 mRNA.

FIG. 15A depicts the alignment of ATF6, XBP1, CREB, and ERSE DNA sequence motifs. The entire oligonucleotide sequence used to construct the 5× ATF6 reporter is shown. The 5′ sequence located outside of the boxed region is the fixed flanking sequence used to generate random oligonucleotides (Wang et al., 2000).

FIG. 15B depicts a schematic representation of unspliced and spliced forms of the murine Xbp1 mRNA and protein coding regions. The translated portion of the two open reading frames, the 26 bp intron, and the bZIP domains are depicted.

FIG. 15C depicts the predicted mRNA secondary structure at the splice site junctions in Xbp1 mRNA. The 3 residues important for cleavage of HAC1 mRNA by IRE1p (−1G, −3C, and +3G) are conserved in the 5′ and 3′ loops.

FIG. 15D depicts a RT-PCR analysis of Xbp1 mRNA splicing using RNA templates from tunicamycin-treated wild-type and IRE1α-null MEFs.

FIG. 15E depicts a Western blot analysis of XBP1. Cell extracts were prepared from wild-type and IRE1α-null MEFs cultured in the presence or absence of tunicamycin (10 μg/ml) with MG132 (10 μM) for increasing times as indicated.

FIG. 15F depicts a Northern blot analysis of Xbp1 mRNA in IRE1α-null MEFs. Wild-type and IRE1α-null MEFs were treated with or without 10 μg/ml tunicamycin for 6 hr prior to harvesting total RNA for Northern blot analysis. The blots were probed with [α-³²P]-labeled 0.94-kb XhoI fragment of XBP1-u and [β-actin cDNA. Quantification of the results showed 3.1- and 4.0-fold induction with tunicamycin treatment in wild-type and IRE1α-null MEFs, respectively.

FIG. 15G depicts a Western blot analysis of XBP1. The 5× ATF6 reporter plasmid and β-galactosidase under control of the CMV promoter were cotransfected into COS-1 cells in the presence of CMV-promoter-driven unspliced form of Xbp1 (XBP1-u), spliced form of Xbp1 (XBP1-s), or the 1st ORF of Xbp1 (XBP1-ORF1) as indicated. Cells were treated with or without tunicamycin (2 μg/ml) for 8 hours before harvest. Proteasome inhibitor (lactacystin, 10 μM) was added to the media for the final 2 hours. An XBP1-reactive polypeptide likely derived from using a cryptic 3′ splice site is indicated by an asterisk.

FIG. 15H demonstrates that 5× ATF6 reporter is activated by IRE1α-dependent Xbp1 mRNA splicing. Wild-type and IRE1α-null MEFs were transfected and assayed as described in FIG. 13 in the presence of CMV-promoter-driven unspliced form of Xbp1 (XBP1-u), spliced form of Xbp1 (XBP1-s), or the 1st ORF of Xbp1 (XBP1-ORF1).

FIGS. 16A-16B demonstrate that IRE1α cleaves both splice site junctions in Xbp1 RNA in vitro and is localized to the inner nuclear envelope.

FIG. 16A depicts [³²P]-labeled wild-type and mutant Xbp1 RNAs, which were prepared and incubated with immunoprecipitated wild-type or RNase-defective (K907A) IRE1α protein in nuclease buffer and analyzed by electrophoresis on a denaturing polyacrylamide gel. The 5′ exon (114 nt), intron (26 nt) and 3′ exon (305 nt) cleavage products of the substrate are marked on the left. The numbers on the right are the expected nucleotide sizes.

FIG. 16B depicts intracellular localization of IRE1α. Wild-type and IRE1α-null MEFs were fractionated as described under “Materials and Methods”. Western blot analysis was performed with mouse anti-IRE1α, human anti-lamin B receptor or rabbit anti-calreticulin antibodies. Lane 1, cellular extract; lane 2, nuclei with inner nuclear membrane; lane 3, Triton X-100 soluble, microsomal and outer nuclear membrane fraction.

FIGS. 17A-17E demonstrate that IRE1α-mediated induction of UPR genes requires ATF6 cleavage.

FIGS. 17A, 17B and 17C depict BiP reporter gene (A and C) and 5× ATF6 reporter gene (B and C) expression in S2P-deficient CHO cells. The reporter plasmids containing the luciferase gene under control of rat BiP promoter or the 5× ATF6 binding sites were cotransfected with β-galactosidase under control of the CMV promoter and an IRE1α (A and B) or ATF6 (C) expression vector into S2P-deficient CHO cells. Immunoglobulin μ heavy chain (μ) and mutant immunoglobulin μ heavy chain deleted of the signal peptide (Δsμ) were used as a positive and negative ER stress inducers, respectively. At 32 hours post-transfection cells were treated with 2 μg/ml tunicamycin for 16 hours prior to harvest. The luciferase activities are presented relative to CMV β-galactosidase activities.

FIG. 17D depicts a Western blot analysis of BiP. Wild-type and S2P-deficient CHO cells were transfected with plasmids as indicated. At 32 hours post-transfection, the transfected cells were treated with 2 μg/ml tunicamycin for 16 hours, harvested, and analyzed by Western blot analysis using anti-BiP antibody.

FIG. 17E depicts a Northern blot analysis of BiP and Xbp1 mRNA in S2P-deficient cells. Wild-type and S2P-deficient CHO cells were treated with or without 2 μg/ml tunicamycin for 16 hours prior to harvesting total RNA for Northern blot analysis using hamster BiP, XBP1-u and β-actin cDNA as probes. Quantification of the results showed tunicamycin induced BiP mRNA 34- and 2.6-fold and Xbp1 mRNA 3.1- and 2.9-fold in wild-type and S2P-deficient CHO cells, respectively.

FIG. 18 demonstrates that ATF6- and IRE1α-dependent UPR signaling pathways merge through regulation of the quantity and quality, respectively, of XBP1 protein. The model depicts the activation of two proximal sensors of the UPR, ATF6 and IRE1α upon ER stress. Upon accumulation of unfolded proteins in the ER lumen, ATF6 leaves the ER to enter the Golgi apparatus where it is cleaved by S1P and then S2P to release a 50 kDa fragment that enters the nucleus through the nuclear pore. p50-ATF6 then interacts with ERSE motifs to activate transcription. Simultaneously and independently, the UPR induces dimerization, autophosphorylation, and activation of the RNase activity of IRE1α that is localized at the inner leaflet of the nuclear envelope. Activated IRE1α then initiates splicing of Xbp1 mRNA to generate a potent transcriptional activator XBP1-s that also enters the nuclear pore to activate transcription from ERSE motifs. The status of XBP1-s and p50-ATF6 when bound to the ERSE is not known, but for simplicity they are depicted as heterodimers.

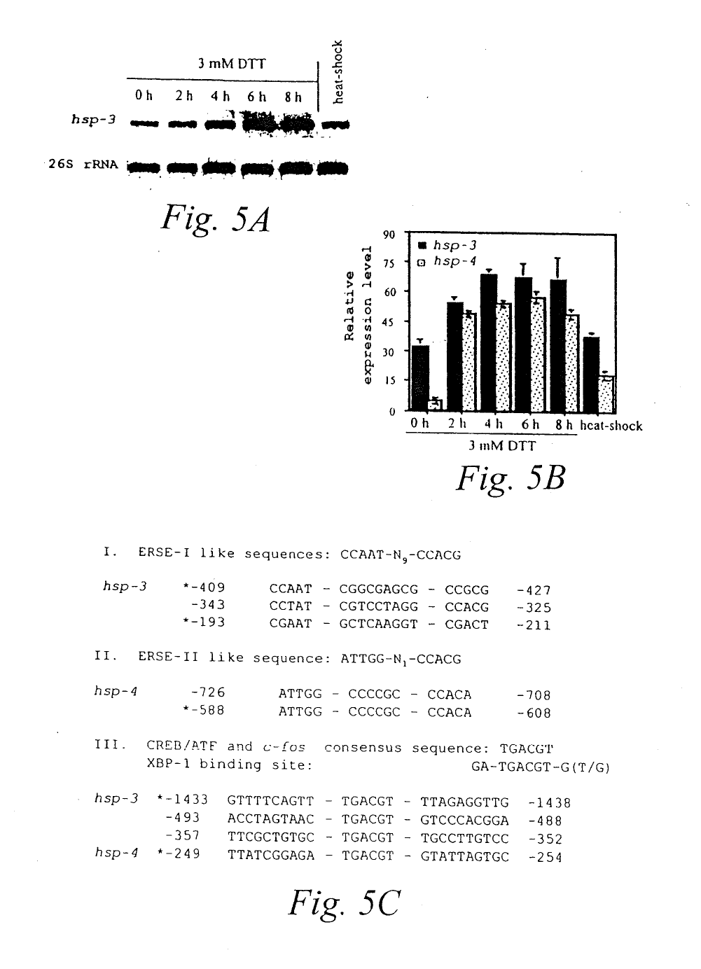

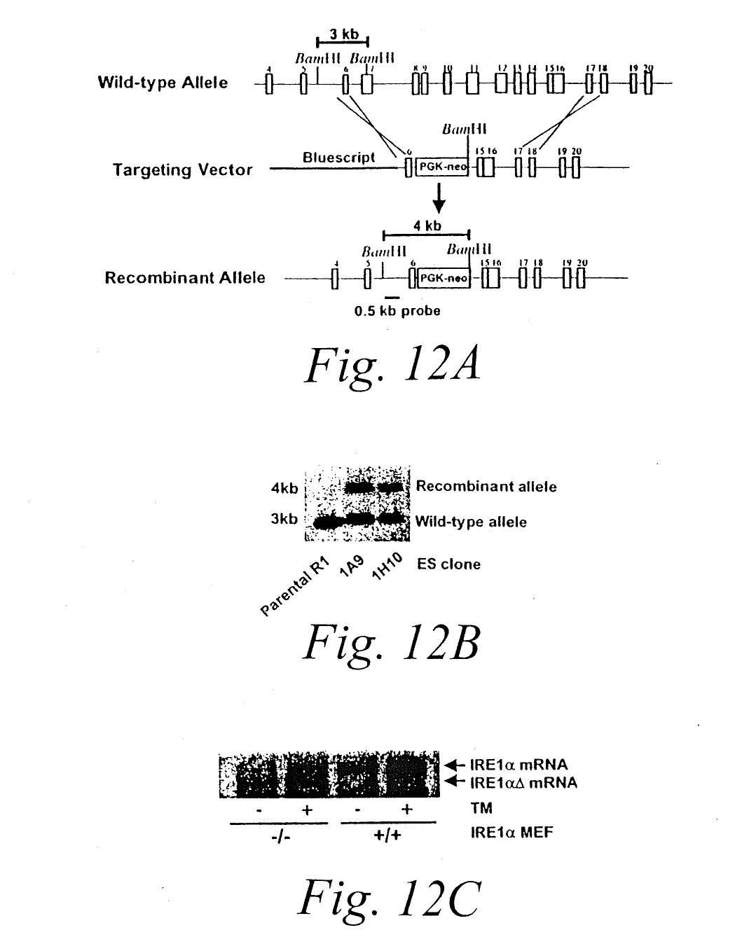

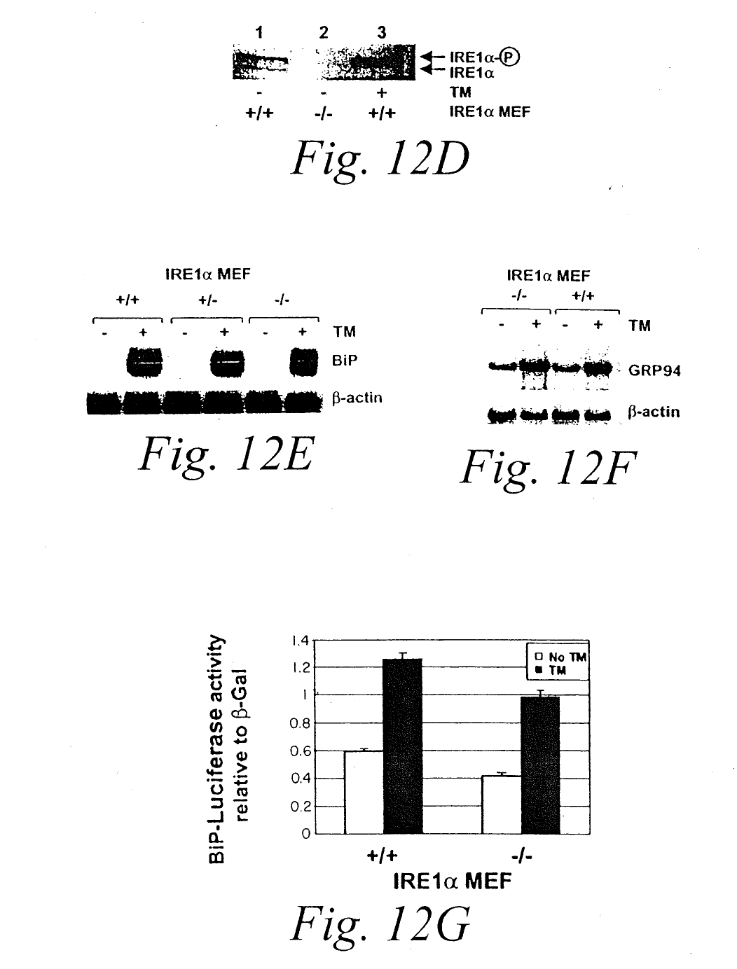

FIGS. 5A-11B may be found in Shen, X. et al. Cell 107:893-903 (2001), and FIGS. 12A-18 may be found in Lee, K. et al. Genes and Develop. 16:452-466 (2002), both of which are expressly incorporated herein by reference.

### DETAILED DESCRIPTION OF THE INVENTION

The present invention relates to methods and compositions for modulating the unfolded protein response. The method further relates to methods and compositions for the treatment and diagnosis of protein conformational diseases or disorders, including, but not limited to, α1-antitrypsin deficiency, cystic fibrosis, and autoimmune diseases and disorders. The invention further provides methods for modulating the unfolded protein response by modulating XBP1 mRNA splicing.

Other features and advantages of the invention will be apparent from the following detailed description and claims.

As used herein, the term “sample” or “biological sample” is intended to include a sample of biological material isolated from a subject, preferably a human subject, or present within a subject, preferably a human subject. The “biological material” can include, for example, tissues, tissue samples, tumors, tumor samples, cells, biological fluids, and purified and/or partially-purified biological molecules. As used herein, the term “isolated”, when used in the context of a biological sample, is intended to indicate that the biological sample has been removed from the subject. In one embodiment, a biological sample comprises a sample which has been isolated from a subject and is subjected to a method of the present invention without further processing or manipulation subsequent to its isolation. In another embodiment, the biological sample can be processed or manipulated subsequent to being isolated and prior to being subjected to a method of the invention. For example, a sample can be refrigerated (e.g., stored at 4° C.), frozen (e.g., stored at −20° C., stored at −135° C., frozen in liquid nitrogen, or cryopreserved using any one of many standard cryopreservation techniques known in the art). Furthermore, a sample can be purified subsequent to isolation from a subject and prior to subjecting it to a method of the present invention. As used herein, the term “purified” when used in the context of a biological sample, is intended to indicate that at least one component of the isolated biological sample has been removed from the biological sample such that fewer components, and consequently, purer components, remain following purification. For example, a serum sample can be separated into one or more components using centrifugation techniques known in the art to obtain partially-purified sample preparation. Furthermore, it is possible to purify a biological sample such that substantially only one component remains. For example, a tissue or tumor sample can be purified such that substantially only the protein or mRNA component of the biological sample remains.

As used herein, the term “protein conformational disease or disorder” includes a disease, disorder or condition associated with the accumulation of unfolded proteins in the endoplasmic reticulum. Examples of protein conformational diseases or disorders include, cystic fibrosis, α1-antitrypsin deficiency and autoimmune diseases and disorders.

The term protein conformational disease or disorder, as used herein, also includes conditions or disorders which are secondary to such disorders, i.e., are influenced or caused by a disorder.

As used interchangeably herein, the terms “XBP1 activity,” “biological activity of XBP1” or “functional activity of XBP1,” include an activity exerted by a spliced XBP1 protein, polypeptide or nucleic acid molecule on a XBP1 responsive cell or tissue or on a XBP1 protein substrate, as determined in vivo, or in vitro, according to standard techniques. XBP1 activity can be a direct activity, such as an association with a XBP1-target molecule. As used herein, a “substrate” or “target molecule” or “binding partner” is a molecule with which a XBP1 protein binds or interacts in nature, such that XBP1-mediated function is achieved. A XBP1 target molecule can be a non-XBP1 molecule (a cofactor or a biochemical molecule involved in a protein conformational disease or disorder), or a XBP1 protein or polypeptide. Examples of such target molecules include proteins in the same signaling path as the XBP1 protein, e.g., proteins which may function upstream (including both stimulators and inhibitors of activity) or downstream of the XBP1 protein in a protein conformational disease or disorder. Alternatively, XBP1 activity is an indirect activity mediated by interaction of the XBP1 protein with a XBP1 target molecule. The biological activities of XBP1 are described herein. For example, the XBP1 molecules of the present invention can have one or more of the following activities: (1) they modulate the unfolded protein response in the endoplasmic reticulum; and (2) they modulate a protein conformational disease or disorder in a subject.

Various aspects of the invention are described in further detail in the following subsections:

### I. Screening Assays:

The invention provides methods (also referred to herein as “screening assays”) for identifying modulators, i.e., candidate or test compounds or agents (e.g., peptides, peptidomimetics, small molecules, ribozymes, or XBP1 antisense molecules) which splice XBP1 mRNA, bind to XBP1 proteins, have a stimulatory or inhibitory effect on XBP1 expression or XBP1 activity, or have a stimulatory or inhibitory effect on the expression or activity of a XBP1 target molecule. Compounds identified using the assays described herein may be useful for treating protein conformational diseases or disorders.

Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. 87:6378-6382; Felici (1991) J. Mol. Biol. 222:301-310; Ladner supra.).

In one aspect, an assay is a cell-based assay in which a cell which expresses a XBP1 protein or biologically active portion thereof is contacted with a test compound and the ability of the test compound to modulate XBP1 activity is determined. The cell, for example, can be of mammalian origin.

In one aspect of the invention, XBP1 activity may be measured by taking aliquots of tissue samples and incubating with 0.1 μM Boc-Phe-Ser-Arg-MCA (Peptide Inc., Osaka, Japan) in 200 μl 0.2M Tris-HCl, pH 8.0 at 37° C. (Okui, A. et al. (2001) Neurochemistry 12:1345-1350). The fluorescence activity may be measured at 380/460 nm every hour. Furthermore, the tissue samples are electrophoresed on a gelatin copolymerized SDS-PAGE under non-reduced conditions, following incubation at 37° C. for 20 hours in 0.1M Tris-HCl, pH 8.0. Subsequently, the gelatin gel is stained with CBB.

The ability of the test compound to modulate XBP1 binding to a substrate can also be determined. Determining the ability of the test compound to modulate XBP1 binding to a substrate can be accomplished, for example, by coupling the XBP1 substrate with a radioisotope, fluorescent, or enzymatic label such that binding of the XBP1 substrate to XBP1 can be determined by detecting the labeled XBP1 substrate in a complex. Alternatively, XBP1 could be coupled with a radioisotope or enzymatic label to monitor the ability of a test compound to modulate XBP1 binding to a XBP1 substrate in a complex. Determining the ability of the test compound to bind XBP1 can be accomplished, for example, by coupling the compound with a radioisotope or enzymatic label such that binding of the compound to XBP1 can be determined by detecting the labeled XBP1 compound in a complex. For example, XBP1 substrates can be labeled with ¹²⁵I, ³⁵S, ¹⁴C, or ³H, either directly or indirectly, and the radioisotope detected by direct counting of radioemission or by scintillation counting. Alternatively, compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound to interact with XBP1 without the labeling of any of the interactants. For example, a microphysiometer can be used to detect the interaction of a compound with) XBP1 without the labeling of either the compound or the XBP1 (McConnell, H. M. et al. (1992) Science 257:1906-1912). As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and XBP1.

To determine whether a test compound modulates XBP1 expression, a cell which expresses XBP1 is contacted with a test compound, and the ability of the test compound to modulate XBP1 expression can be determined by measuring XBP1 mRNA by, e.g., Northern Blotting, quantitative PCR (e.g., TaqMan), or in vitro transcriptional assays. To perform an in vitro transcriptional assay, the full length promoter and enhancer of XBP1 can be linked to a reporter gene such as chloramphenicol acetyltransferase (CAT) or luciferase and introduced into host cells. The same host cells can then be transfected with or contacted with the test compound. The effect of the test compound can be measured by reporter gene activity and comparison to reporter gene activity in cells which do not contain the test compound. An increase or decrease in reporter gene activity indicates a modulation of XBP1 expression.

In yet another embodiment, an assay of the present invention is a cell-free assay in which a XBP1 protein or biologically active portion thereof is contacted with a test compound and the ability of the test compound to bind to or to modulate (e.g., stimulate or inhibit) the activity of the XBP1 protein or biologically active portion thereof is determined. Preferred biologically active portions of the XBP1 proteins to be used in assays of the present invention include fragments that participate in interactions with non-XBP1 molecules. Binding of the test compound to the XBP1 protein can be determined either directly or indirectly as described above. Determining the ability of the XBP1 protein to bind to a test compound can also be accomplished using a technology such as real-time Biomolecular Interaction Analysis (BIA) (Sjolander, S. and Urbaniczky, C. (1991) Anal. Chem. 63:2338-2345; Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting a XBP1 protein or biologically active portion thereof with a known compound which binds the XBP1 protein to form an assay mixture, contacting the assay mixture with a test compound, and determining the ability of the test compound to interact with the XBP1 protein, wherein determining the ability of the test compound to interact with the XBP1 protein comprises determining the ability of the XBP1 protein to preferentially bind to or modulate the activity of a XBP1 target molecule (e.g., a XBP1 substrate).

The cell-free assays of the present invention are amenable to use of both soluble and/or membrane-bound forms of isolated proteins (e.g., XBP1 proteins or biologically active portions thereof). In the case of cell-free assays in which a membrane-bound form of an isolated protein is used it may be desirable to utilize a solubilizing agent such that the membrane-bound form of the isolated protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either XBP1 or a XBP1 target molecule to facilitate separation of complexed from uncomplexed forms of one or both of the proteins, as well as to accommodate automation of the assay. Binding of a test compound to a XBP1 protein, or interaction of a XBP1 protein with a XBP1 target molecule in the presence and absence of a test compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/XBP1 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-absorbed target protein or XBP1 protein, and the mixture incubated under conditions conducive to complex formation (e.g., at physiological conditions for salt and pH). Following incubation, the beads or microtitre plate wells are washed to remove any unbound components, the matrix is immobilized in the case of beads, and complex formation is determined either directly or indirectly, for example, as described above. Alternatively, the complexes can be dissociated from the matrix, and the level of XBP1 binding or activity determined using standard techniques.

Other techniques for immobilizing proteins or cell membrane preparations on matrices can also be used in the screening assays of the invention. For example, either a) XBP1 protein or a XBP1 target molecule can be immobilized utilizing conjugation of biotin and streptavidin. Biotinylated XBP1 protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies which are reactive with XBP1 protein or target molecules but which do not interfere with binding of the XBP1 protein to its target molecule can be derivatized to the wells of the plate, and unbound target or XBP1 protein is trapped in the wells by antibody conjugation. Methods for detecting such complexes, in addition to those described above for the GST-immobilized complexes, include immunodetection of complexes using antibodies reactive with the XBP1 protein or target molecule, as well as enzyme-linked assays which rely on detecting an enzymatic activity associated with the XBP1 protein or target molecule.

In yet another aspect of the invention, the XBP1 protein or fragments thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300) to identify other proteins which bind to or interact with XBP1 (“XBP1-binding proteins” or “XBP1-bp”) and are involved in XBP1 activity. Such XBP1-binding proteins are also likely to be involved in the propagation of signals by the XBP1 proteins or XBP1 targets as, for example, downstream elements of a XBP1-mediated signaling pathway. Alternatively, such XBP1-binding proteins are likely to be XBP1 inhibitors.

The two-hybrid system is based on the modular nature of most transcription factors, which consist of separable DNA-binding and activation domains. Briefly, the assay utilizes two different DNA constructs. In one construct, the gene that codes for a) XBP1 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a XBP1-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene that encodes the protein that interacts with the XBP1 protein.

In another aspect, the invention pertains to a combination of two or more of the assays described herein. For example, a modulating agent can be identified using a cell-based or a cell-free assay, and the ability of the agent to modulate the activity of a XBP1 protein can be confirmed in vivo.

Moreover, a XBP1 modulator identified as described herein (e.g., an antisense XBP1 nucleic acid molecule, a XBP1-specific antibody, or a small molecule) can be used in an animal model to determine the efficacy, toxicity, or side effects of treatment with such a modulator. Alternatively, a XBP1 modulator identified as described herein can be used in an animal model to determine the mechanism of action of such a modulator.

### II. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining XBP1 mRNA splicing, XBP1 protein and/or nucleic acid expression, as well as XBP1 activity, in the context of a biological sample (e.g., blood, serum, cells, or tissue) to thereby determine whether an individual is afflicted with a protein conformational disease or disorder. The invention also provides for prognostic (or predictive) assays for determining whether an individual is at risk of developing a protein conformational disease or disorder. For example, mutations in a XBP1 gene can be assayed for in a biological sample. Such assays can be used for prognostic or predictive purpose to thereby prophylactically treat an individual prior to the onset of a protein conformational disease or disorder.

Another aspect of the invention pertains to monitoring the influence of XBP1 modulators (e.g., anti-XBP1 antibodies or XBP1 ribozymes) on the expression or activity of XBP1 in clinical trials.

These and other agents are described in further detail in the following sections.

A. Diagnostic Assays for Protein Conformational Diseases or Disorders

To determine whether a subject is afflicted with a protein conformational disease or disorder, a biological sample may be obtained from a subject and the biological sample may be contacted with a compound or an agent capable of detecting a spliced) XBP1 mRNA molecule, a XBP1 protein or nucleic acid (e.g., mRNA or genomic DNA) that encodes a XBP1 protein, in the biological sample. A preferred agent for detecting spliced XBP1 mRNA or genomic DNA is a labeled nucleic acid probe capable of hybridizing to spliced XBP1 mRNA or genomic DNA. The nucleic acid probe can be, for example, the spliced XBP1 mRNA molecule set forth in SEQ ID NO:1, or a portion thereof, such as an oligonucleotide of at least 15, 20, 25, 30, 25, 40, 45, 50, 100, 250 or 500 nucleotides in length and sufficient to specifically hybridize under stringent conditions to spliced XBP1 mRNA or genomic DNA. Other suitable probes for use in the diagnostic assays of the invention are described herein.

A preferred agent for detecting XBP1 protein in a sample is an antibody capable of binding to XBP1 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of direct substances that can be coupled to an antibody or a nucleic acid probe include various enzymes, prosthetic groups, fluorescent materials, luminescent materials, bioluminescent materials, and radioactive materials. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect spliced XBP1 mRNA, XBP1 protein, or genomic DNA in a biological sample in vitro as well as in vivo. For example, in vitro techniques for detection of spliced XBP1 mRNA include Northern hybridizations and in situ hybridizations. In vitro techniques for detection of XBP1 protein include enzyme linked immunosorbent assays (ELISAs), Western blots, immunoprecipitations and immunofluorescence. In vitro techniques for detection of) XBP1 genomic DNA include Southern hybridizations. Furthermore, in vivo techniques for detection of XBP1 protein include introducing into a subject a labeled anti-XBP1 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting XBP1 protein, spliced XBP1 mRNA, or genomic DNA, such that the presence of XBP1 protein, spliced XBP1 mRNA or genomic DNA is detected in the biological sample, and comparing the presence of XBP1 protein, spliced XBP1 mRNA or genomic DNA in the control sample with the presence of XBP1 protein, spliced XBP1 mRNA or genomic DNA in the test sample.

B. Prognostic Assays for Protein Conformational Diseases or Disorders

The present invention further pertains to methods for identifying subjects having or at risk of developing a protein conformational disease or disorder, e.g., a protein conformational disease or disorder associated with aberrant XBP1 expression or activity.

As used herein, the term “aberrant” includes a XBP1 expression or activity that deviates from the wild type XBP1 expression or activity. Aberrant expression or activity includes increased or decreased expression or activity, as well as expression or activity that does not follow the wild type developmental pattern of expression or the subcellular pattern of expression. For example, aberrant XBP1 expression or activity is intended to include the cases in which a mutation in the XBP1 gene causes the XBP1 gene to be under-expressed or over-expressed and situations in which such mutations result in a non-functional XBP1 protein or a protein which does not function in a wild-type fashion, e.g., a protein which does not interact with a XBP1 substrate, or one which interacts with a non-XBP1 substrate.

The assays described herein, such as the preceding diagnostic assays or the following assays, can be used to identify a subject having or at risk of developing a protein conformational disease or disorder, e.g, α1-antitrypsin deficiency, cystic fibrosis and autoimmune diseases and disorders. A biological sample may be obtained from a subject and tested for the presence or absence of a genetic alteration. For example, such genetic alterations can be detected by ascertaining the existence of at least one of 1) a deletion of one or more nucleotides from a XBP1 gene, 2) an addition of one or more nucleotides to a XBP1 gene, 3) a substitution of one or more nucleotides of a XBP1 gene, 4) a chromosomal rearrangement of a XBP1 gene, 5) an alteration in the level of a messenger RNA transcript of a XBP1 gene, 6) aberrant modification of a XBP1 gene, such as of the methylation pattern of the genomic DNA, 7) the presence of a non-wild type splicing pattern of a messenger RNA transcript of a XBP1 gene, 8) a non-wild type level of a XBP1-protein, 9) allelic loss of a XBP1 gene, and 10) inappropriate post-translational modification of a XBP1-protein.

As described herein, there are a large number of assays known in the art that can be used for detecting genetic alterations in a XBP1 gene. For example, a genetic alteration in a XBP1 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a XBP1 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a XBP1 gene under conditions such that hybridization and amplification of the XBP1 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a XBP1 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in XBP1 can be identified by hybridizing biological sample derived and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in XBP1 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the cDNA from spliced XBP1 mRNA in a biological sample and detect mutations by comparing the sequence of the XBP1 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the XBP1 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type XBP1 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in XBP1 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a XBP1 sequence, e.g., a wild-type XBP1 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in XBP1 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control XBP1 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a XBP1 modulator (e.g., an agonist, antagonist, peptidomimetic, protein, peptide, nucleic acid, or small molecule) to effectively treat a pain.

C. Monitoring of Effects During Clinical Trials

The present invention further provides methods for determining the effectiveness of a XBP1 modulator (e.g., a XBP1 modulator identified herein) in treating a protein conformational disease or disorder in a subject. For example, the effectiveness of a XBP1 modulator in increasing XBP1 mRNA splicing, increasing XBP1 gene expression, protein levels, or in upregulating XBP1 activity, can be monitored in clinical trials of subjects exhibiting decreased XBP1 mRNA splicing, XBP1 gene expression, protein levels, or downregulated XBP1 activity. Alternatively, the effectiveness of a XBP1 modulator in decreasing XBP1 gene expression, protein levels, or in downregulating XBP1 activity, can be monitored in clinical trials of subjects exhibiting increased XBP1 mRNA splicing, increased XBP1 gene expression, protein levels, or XBP1 activity. In such clinical trials, the expression or activity of a XBP1 gene, and preferably, other genes that have been implicated in, for example, a protein conformational disease or disorder can be used as a “read out” or marker of the phenotype of a particular cell.

For example, and not by way of limitation, genes, including XBP1, that are modulated in cells by treatment with an agent which modulates XBP1 activity (e.g., identified in a screening assay as described herein) can be identified. Thus, to study the effect of agents which modulate XBP1 activity on subjects suffering from a protein conformational disease or disorder in, for example, a clinical trial, cells can be isolated and RNA prepared and analyzed for the levels of expression of XBP1 and other genes implicated in the protein conformational disease or disorder. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of XBP1 or other genes. In this way, the gene expression pattern can serve as a marker, indicative of the physiological response of the cells to the agent which modulates XBP1 activity. This response state may be determined before, and at various points during treatment of the individual with the agent which modulates XBP1 activity.

In a preferred embodiment, the present invention provides a method for monitoring the effectiveness of treatment of a subject with an agent that modulates XBP1 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a XBP1 protein, spliced XBP1 mRNA, or genomic DNA in the pre-administration sample; (iii) obtaining one or more post-administration samples from the subject; (iv) detecting the level of expression or activity of the XBP1 protein, spliced XBP1 mRNA, or genomic DNA in the post-administration samples; (v) comparing the level of expression or activity of the XBP1 protein, spliced XBP1 mRNA, or genomic DNA in the pre-administration sample with the XBP1 protein, spliced XBP1 mRNA, or genomic DNA in the post administration sample or samples; and (vi) altering the administration of the agent to the subject accordingly. For example, increased administration of the agent may be desirable to increase the expression or activity of XBP1 to higher levels than detected, i.e., to increase the effectiveness of the agent. Alternatively, decreased administration of the agent may be desirable to decrease expression or activity of XBP1 to lower levels than detected, i.e. to decrease the effectiveness of the agent. According to such an embodiment, XBP1 expression or activity may be used as an indicator of the effectiveness of an agent, even in the absence of an observable phenotypic response.

III. Methods of Treatment of Subjects Suffering from Protein Conformational Diseases or Disorders:

The present invention provides for both prophylactic and therapeutic methods of treating a subject, e.g., a human, at risk of (or susceptible to) a protein conformational disease or disorder such as α1-antitrypsin deficiency, cystic fibrosis, and autoimmune diseases and disorders. As used herein, “treatment” of a subject includes the application or administration of a therapeutic agent to a subject, or application or administration of a therapeutic agent to a cell or tissue from a subject, who has a disease or disorder, has a symptom of a disease or disorder, or is at risk of (or susceptible to) a disease or disorder, with the purpose to cure, heal, alleviate, relieve, alter, remedy, ameliorate, improve, or affect the disease or disorder, the symptom of the disease or disorder, or the risk of (or susceptibility to) the disease or disorder. As used herein, a “therapeutic agent” includes, but is not limited to, small molecules, peptides, polypeptides, antibodies, ribozymes, and antisense oligonucleotides.

With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring a subject's prophylactic or therapeutic treatment with either the XBP1 molecules of the present invention or XBP1 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a protein conformational disease or disorder by administering to the subject an agent which modulates XBP1 expression or XBP1 activity, in a cell. Subjects at risk for developing a protein conformational disease or disorder can be identified by, for example, any or a combination of the diagnostic or prognostic assays described herein. Administration of a prophylactic agent can occur prior to the manifestation of symptoms characteristic of aberrant XBP1 expression or activity, such that a protein conformational disease or disorder is prevented or, alternatively, delayed in its progression. Depending on the type of XBP1 aberrancy, for example, a XBP1 molecule, XBP1 agonist or XBP1 antagonist agent can be used for treating the subject. The appropriate agent can be determined based on screening assays described herein.

B. Therapeutic Methods

Another aspect of the invention pertains to methods for treating a subject suffering from a protein conformational disease or disorder. These methods involve administering to a subject an agent which modulates XBP1 expression or activity (e.g., an agent identified by a screening assay described herein), or a combination of such agents. In another embodiment, the method involves administering to a subject a XBP1 protein or nucleic acid molecule as therapy to compensate for reduced, aberrant, or unwanted XBP1 expression or activity.

Stimulation of XBP1 activity is desirable in situations in which XBP1 is abnormally downregulated and/or in which increased XBP1 activity is likely to have a beneficial effect. Likewise, inhibition of XBP1 activity is desirable in situations in which XBP1 is abnormally upregulated and/or in which decreased XBP1 activity is likely to have a beneficial effect.

The agents which modulate XBP1 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates XBP1 activity (e.g., a fragment of a XBP1 protein or an anti-XBP1 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate XBP1 activity can also be prepared in the form of suppositories (e.g., with conventional suppository bases such as cocoa butter and other glycerides) or retention enemas for rectal delivery.

In one embodiment, the agents that modulate XBP1 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates XBP1 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such XBP1 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg pain, preferably about 0.01 to 25 mg/kg pain, more preferably about 0.1 to 20 mg/kg pain, and even more preferably about 1 to 10 mg/kg, 2 to 9 mg/kg, 3 to 8 mg/kg, 4 to 7 mg/kg, or 5 to 6 mg/kg pain. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg pain, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate splicing of XBP1 mRNA, XBP1 expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a XBP1 polypeptide or nucleic acid molecule, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, pain, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

C. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates XBP1 activity, as well as tailoring the dosage and/or therapeutic regimen of treatment with an agent which modulates XBP1 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a XBP1 protein of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a XBP1 molecule or XBP1 modulator of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a protein conformational disease or disorder with an agent which modulates XBP1 activity.

### IV. Recombinant Expression Vectors and Host Cells Used in the Methods of the Invention

The methods of the invention (e.g., the screening assays described herein) include the use of vectors, preferably expression vectors, containing a nucleic acid encoding a XBP1 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., XBP1 proteins, mutant forms of XBP1 proteins, fusion proteins, and the like).

The recombinant expression vectors to be used in the methods of the invention can be designed for expression of XBP1 proteins in prokaryotic or eukaryotic cells. For example, XBP1 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in XBP1 activity assays, (e.g., direct assays or competitive assays described in detail below), or to generate antibodies specific for XBP1 proteins. In a preferred embodiment, a XBP1 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to spliced XBP1 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1(1) 1986.

Another aspect of the invention pertains to the use of host cells into which a XBP1 nucleic acid molecule of the invention is introduced, e.g., a XBP1 nucleic acid molecule within a recombinant expression vector or a XBP1 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a XBP1 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a XBP1 protein. Accordingly, the invention further provides methods for producing a XBP1 protein using the host cells of the invention. In one embodiment, the method comprises culturing the host cell of the invention (into which a recombinant expression vector encoding a XBP1 protein has been introduced) in a suitable medium such that a XBP1 protein is produced. In another embodiment, the method further comprises isolating a XBP1 protein from the medium or the host cell.

### V. Isolated Nucleic Acid Molecules Used in the Methods of the Invention

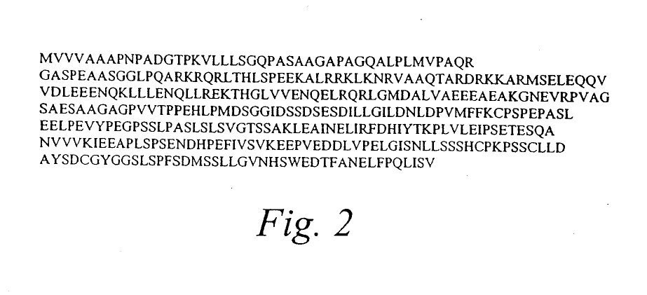

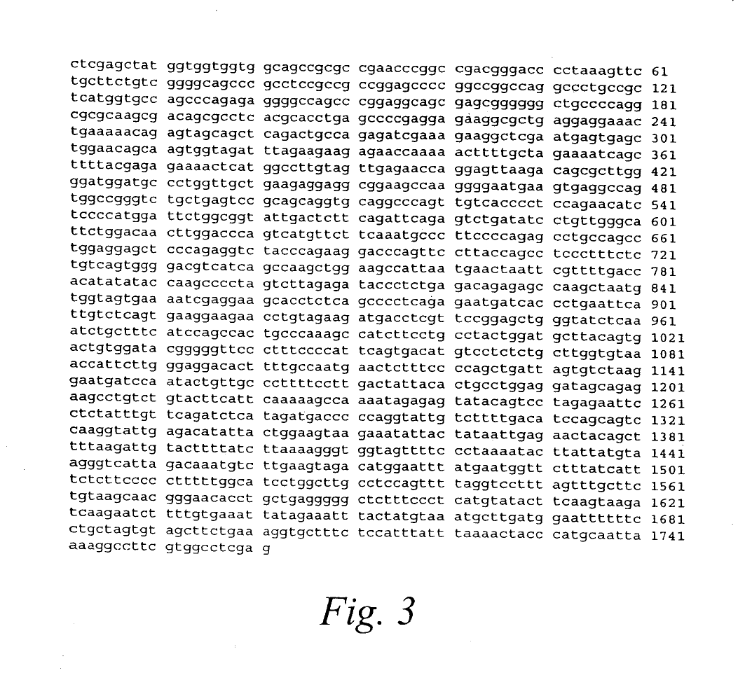

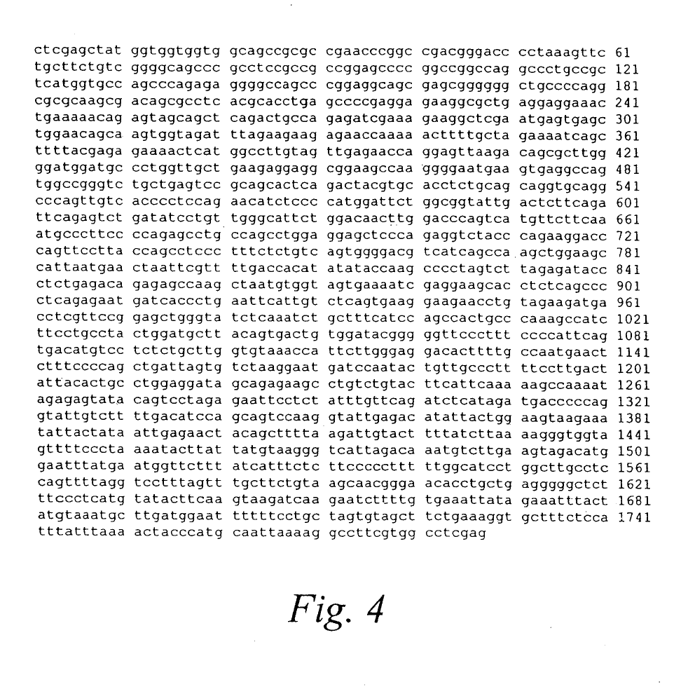

The cDNA sequence of the isolated human XBP1 gene and the predicted amino acid sequence of the human XBP1 polypeptide are shown in SEQ ID NOs:3 and 2, respectively, and in FIGS. 3 and 2. The cDNA sequence of the human unspliced XBP1 is shown in SEQ ID NO:4 and in FIG. 4.

The methods of the invention include the use of isolated nucleic acid molecules that encode XBP1 proteins or biologically active portions thereof, as well as nucleic acid fragments sufficient for use as hybridization probes to identify XBP1-encoding nucleic acid molecules (e.g., spliced XBP1 mRNA) and fragments for use as PCR primers for the amplification or mutation of XBP1 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, or a portion thereof, can be isolated using standard molecular biology techniques and the sequence information provided herein. Using all or portion of the nucleic acid sequence of SEQ ID NO:1 as a hybridization probe, XBP1 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1 can be isolated by the polymerase chain reaction (PCR) using synthetic oligonucleotide primers designed based upon the sequence of SEQ ID NO:1.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to XBP1 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, a complement of the nucleotide sequence shown in SEQ ID NO:1, or a portion of any of these nucleotide sequences. A nucleic acid molecule which is complementary to the nucleotide sequence shown in SEQ ID NO:1, is one which is sufficiently complementary to the nucleotide sequence shown in SEQ ID NO:1 such that it can hybridize to the nucleotide sequence shown in SEQ ID NO:1 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or more identical to the entire length of the nucleotide sequence shown in SEQ ID NO:1, or a portion of any of this nucleotide sequence.

Moreover, the nucleic acid molecules used in the methods of the invention can comprise only a portion of the nucleic acid sequence of SEQ ID NO:1, for example, a fragment which can be used as a probe or primer or a fragment encoding a portion of a XBP1 protein, e.g., a biologically active portion of a XBP1 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1 or an anti-sense sequence of SEQ ID NO:1. In one embodiment, a nucleic acid molecule used in the methods of the present invention comprises a nucleotide sequence which is greater than 50, 50-100, 100-200, 200-300, 300-400, 400-500, 500-600, 600-700, 700-800, 800-900, 900-1000, 1000-1100 or more nucleotides in length and hybridizes under stringent hybridization conditions to a nucleic acid molecule of SEQ ID NO:1.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology,

Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× or 6× sodium chloride/sodium citrate (SSC), at about 65-70° C. (or hybridization in 4×SSC plus 50% formamide at about 42-50° C.) followed by one or more washes in 1×SSC, at about 65-70° C. A further preferred, non-limiting example of stringent hybridization conditions includes hybridization at 6×SSC at 45° C., followed by one or more washes in 0.2×SSC, 0.1% SDS at 65° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×or 6×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases) +4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a XBP1 protein, such as by measuring a level of a XBP1-encoding nucleic acid in a sample of cells from a subject e.g., detecting spliced XBP1 mRNA levels or determining whether a genomic XBP1 gene has been mutated or deleted.

The methods of the invention further encompass the use of nucleic acid molecules that differ from the nucleotide sequence shown in SEQ ID NO:1 due to degeneracy of the genetic code and thus encode the same XBP1 proteins as those encoded by the nucleotide sequence shown in SEQ ID NO:1. In another embodiment, an isolated nucleic acid molecule included in the methods of the invention has a nucleotide sequence encoding a protein having an amino acid sequence shown in SEQ ID NO:2.

The methods of the present invention may further use non-human orthologues of the human XBP1 protein. Orthologues of the human XBP1 protein are proteins that are isolated from non-human organisms and possess the same XBP1 activity.

The methods of the present invention further include the use of nucleic acid molecules comprising the nucleotide sequence of SEQ ID NO:1, or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of XBP1 (e.g., the sequence of SEQ ID NO:2) without altering the biological activity, whereas an “essential” amino acid residue is required for biological activity. For example, amino acid residues that are conserved among the XBP1 proteins of the present invention and other members of the short-chain dehydrogenase family are not likely to be amenable to alteration.

Mutations can be introduced into SEQ ID NO:1 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a XBP1 protein is preferably replaced with another amino acid residue from the same side chain family. Alternatively, in another embodiment, mutations can be introduced randomly along all or part of a xBP1 coding sequence, such as by saturation mutagenesis, and the resultant mutants can be screened for XBP1 biological activity to identify mutants that retain activity. Following mutagenesis of SEQ ID NO:1, the encoded protein can be expressed recombinantly and the activity of the protein can be determined using an assay described herein.

Another aspect of the invention pertains to the use of isolated nucleic acid molecules which are antisense to the nucleotide sequence of SEQ ID NO:1. An “antisense” nucleic acid comprises a nucleotide sequence which is complementary to a “sense” nucleic acid encoding a protein, e.g., complementary to the coding strand of a double-stranded cDNA from the spliced mRNA molecule or complementary to a spliced XBP1 mRNA sequence. Accordingly, an antisense nucleic acid can hydrogen bond to a sense nucleic acid. The antisense nucleic acid can be complementary to an entire spliced XBP1 coding strand, or to only a portion thereof. In one embodiment, an antisense nucleic acid molecule is antisense to a “coding region” of the coding strand of a nucleotide sequence encoding a XBP1. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding XBP1. The term “noncoding region” refers to 5′ and 3′ sequences which flank the coding region that are not translated into amino acids (also referred to as 5′ and 3′ untranslated regions).

Given the coding strand sequences encoding XBP1 disclosed herein, antisense nucleic acids of the invention can be designed according to the rules of Watson and Crick base pairing. The antisense nucleic acid molecule can be complementary to the entire coding region of spliced XBP1 mRNA, but more preferably is an oligonucleotide which is antisense to only a portion of the coding or noncoding region of spliced XBP1 mRNA. For example, the antisense oligonucleotide can be complementary to the region surrounding the translation start site of XBP1 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules used in the methods of the invention are typically administered to a subject or generated in situ such that they hybridize with or bind to cellular mRNA and/or genomic DNA encoding a XBP1 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haseloff and Gerlach (1988) Nature 334:585-591)) can be used to catalytically cleave XBP1 mRNA transcripts to thereby inhibit translation of XBP1 mRNA. A ribozyme having specificity for a XBP1-encoding nucleic acid can be designed based upon the nucleotide sequence of a XBP1 cDNA disclosed herein (i.e., SEQ ID NO:1). For example, a derivative of a Tetrahymena L-19 IVS RNA can be constructed in which the nucleotide sequence of the active site is complementary to the nucleotide sequence to be cleaved in a XBP1-encoding mRNA. See, e.g., Cech et al. U.S. Pat. No. 4,987,071; and Cech et al. U.S. Pat. No. 5,116,742. Alternatively, XBP1 mRNA can be used to select a catalytic RNA having a specific ribonuclease activity from a pool of RNA molecules. See, e.g., Bartel, D. and Szostak, J. W. (1993) Science 261:1411-1418.

Alternatively, XBP1 gene expression can be inhibited by targeting nucleotide sequences complementary to the regulatory region of the XBP1 (e.g., the XBP1 promoter and/or enhancers) to form triple helical structures that prevent transcription of the XBP1 gene in target cells. See generally, Helene, C. (1991) Anticancer Drug Des. 6(6): 569-84; Helene, C. et al. (1992) Ann. N.Y. Acad. Sci. 660:27-36; and Maher, L. J. (1992) Bioessays 14(12):807-15.

In yet another embodiment, the XBP1 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup, B. and Nielsen, P. E. (1996) Bioorg. Med. Chem. 4(1):5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. and Nielsen (1996) supra and Perry-O'Keefe et al. (1996) Proc. Natl. Acad. Sci. USA 93:14670-675.

PNAs of XBP1 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of XBP1 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup and Nielsen (1996) supra)); or as probes or primers for DNA sequencing or hybridization (Hyrup and Nielsen (1996) supra; Perry-O'Keefe et al. (1996) supra).

In another embodiment, PNAs of XBP1 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of XBP1 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup and Nielsen (1996) supra). The synthesis of PNA-DNA chimeras can be performed as described in Hyrup and Nielsen (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acids Res. 17: 5973-88). PNA monomers are then coupled in a stepwise manner to produce a chimeric molecule with a 5′ PNA segment and a 3′ DNA segment (Finn et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W089/10134). In addition, oligonucleotides can be modified with hybridization-triggered cleavage agents (See, e.g., Krol et al. (1988) Biotechniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

### VI. Isolated XBP1 Proteins and Anti-XBP1 Antibodies Used in the Methods of the Invention

The methods of the invention include the use of isolated XBP1 proteins, and biologically active portions thereof, as well as polypeptide fragments suitable for use as immunogens to raise anti-XBP1 antibodies. In one embodiment, native XBP1 proteins can be isolated from cells or tissue sources by an appropriate purification scheme using standard protein purification techniques. In another embodiment, XBP1 proteins are produced by recombinant DNA techniques. Alternative to recombinant expression, a XBP1 protein or polypeptide can be synthesized chemically using standard peptide synthesis techniques.

As used herein, a “biologically active portion” of a XBP1 protein includes a fragment of a XBP1 protein having a XBP1 activity. Biologically active portions of a XBP1 protein include peptides comprising amino acid sequences sufficiently identical to or derived from the amino acid sequence of the XBP1 protein, e.g., the amino acid sequence shown in SEQ ID NO:2, which include fewer amino acids than the full length XBP1 proteins, and exhibit at least one activity of a XBP I protein. Typically, biologically active portions comprise a domain or motif with at least one activity of the XBP1 protein. A biologically active portion of a XBP1 protein can be a polypeptide which is, for example, 25, 50, 75, 100, 125, 150, 175, 200, 250, 300 or more amino acids in length. Biologically active portions of a XBP1 protein can be used as targets for developing agents which modulate a XBP1 activity.

In a preferred embodiment, the XBP1 protein used in the methods of the invention has an amino acid sequence shown in SEQ ID NO:2. In other embodiments, the XBP1 protein is substantially identical to SEQ ID NO:2, and retains the functional activity of the protein of SEQ ID NO:2, yet differs in amino acid sequence due to natural allelic variation or mutagenesis, as described in detail in subsection V above. Accordingly, in another embodiment, the XBP1 protein used in the methods of the invention is a protein which comprises an amino acid sequence at least about 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% A or more identical to SEQ ID NO:2.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the XBP1 amino acid sequence of SEQ ID NO:2 having 376 amino acids residues, at least 93, preferably at least 124, more preferably at least 156, even more preferably at least 187, and even more preferably at least 200, 250, 300, 350, 375 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use XBP1 chimeric or fusion proteins. As used herein, a XBP1 “chimeric protein” or “fusion protein” comprises a XBP1 polypeptide operatively linked to a non-XBP1 polypeptide. A “XBP1 polypeptide” refers to a polypeptide having an amino acid sequence corresponding to a XBP1 molecule, whereas a “non-XBP1 polypeptide” refers to a polypeptide having an amino acid sequence corresponding to a protein which is not substantially homologous to the XBP1 protein, e.g., a protein which is different from the XBP1 protein and which is derived from the same or a different organism. Within a XBP1 fusion protein the XBP1 polypeptide can correspond to all or a portion of a XBP1 protein. In a preferred embodiment, a XBP1 fusion protein comprises at least one biologically active portion of a XBP1 protein. In another preferred embodiment, a XBP1 fusion protein comprises at least two biologically active portions of a XBP1 protein. Within the fusion protein, the term “operatively linked” is intended to indicate that the XBP1 polypeptide and the non-XBP1 polypeptide are fused in-frame to each other. The non-XBP1 polypeptide can be fused to the N-terminus or C-terminus of the XBP1 polypeptide.

For example, in one embodiment, the fusion protein is a GST-XBP1 fusion protein in which the XBP1 sequences are fused to the C-terminus of the GST sequences. Such fusion proteins can facilitate the purification of recombinant XBP1.

In another embodiment, this fusion protein is a XBP1 protein containing a heterologous signal sequence at its N-terminus. In certain host cells (e.g., mammalian host cells), expression and/or secretion of XBP1 can be increased through use of a heterologous signal sequence.

The XBP1 fusion proteins used in the methods of the invention can be incorporated into pharmaceutical compositions and administered to a subject in vivo. The XBP1 fusion proteins can be used to affect the bioavailability of a XBP1 substrate. Use of XBP1 fusion proteins may be useful therapeutically for the treatment of disorders caused by, for example, (i) aberrant modification or mutation of a gene encoding a) XBP1 protein; (ii) mis-regulation of the XBP1 gene; and (iii) aberrant post-translational modification of a XBP1 protein.

Moreover, the XBP1-fusion proteins used in the methods of the invention can be used as immunogens to produce anti-XBP1 antibodies in a subject, to purify XBP1 ligands and in screening assays to identify molecules which inhibit the interaction of XBP1 with a XBP1 substrate.

Preferably, a XBP1 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A XBP1-encoding nucleic acid can be cloned into such an expression vector such that the fusion moiety is linked in-frame to the XBP1 protein.

The present invention also pertains to the use of variants of the XBP1 proteins which function as either XBP1 agonists (mimetics) or as XBP1 antagonists. Variants of the XBP1 proteins can be generated by mutagenesis, e.g., discrete point mutation or truncation of a XBP1 protein. An agonist of the XBP1 proteins can retain substantially the same, or a subset, of the biological activities of the naturally occurring form of a XBP1 protein. An antagonist of a XBP1 protein can inhibit one or more of the activities of the naturally occurring form of the XBP1 protein by, for example, competitively modulating a XBP1-mediated activity of a XBP1 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the XBP1 protein.

In one embodiment, variants of a XBP1 protein which function as either XBP1 agonists (mimetics) or as XBP1 antagonists can be identified by screening combinatorial libraries of mutants, e.g., truncation mutants, of a XBP1 protein for XBP1 protein agonist or antagonist activity. In one embodiment, a variegated library of XBP1 variants is generated by combinatorial mutagenesis at the nucleic acid level and is encoded by a variegated gene library. A variegated library of XBP1 variants can be produced by, for example, enzymatically ligating a mixture of synthetic oligonucleotides into gene sequences such that a degenerate set of potential XBP1 sequences is expressible as individual polypeptides, or alternatively, as a set of larger fusion proteins (e.g., for phage display) containing the set of XBP1 sequences therein. There are a variety of methods which can be used to produce libraries of potential XBP1 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential XBP1 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a XBP1 protein coding sequence can be used to generate a variegated population of XBP1 fragments for screening and subsequent selection of variants of a XBP1 protein. In one embodiment, a library of coding sequence fragments can be generated by treating a double stranded PCR fragment of a XBP1 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with Si nuclease, and ligating the resulting fragment library into an expression vector. By this method, an expression library can be derived which encodes N-terminal, C-terminal and internal fragments of various sizes of the XBP1 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of) XBP1 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify XBP1 variants (Arkin and Youvan (1992) Proc. Natl. Acad. Sci. USA 89:7811-7815; Delagrave et al. (1993) Prot. Eng. 6(3):327-331).

The methods of the present invention further include the use of anti-XBP1 antibodies. An isolated XBP1 protein, or a portion or fragment thereof, can be used as an immunogen to generate antibodies that bind XBP1 using standard techniques for polyclonal and monoclonal antibody preparation. A full-length XBP1 protein can be used or, alternatively, antigenic peptide fragments of XBP1 can be used as immunogens. The antigenic peptide of XBP1 comprises at least 8 amino acid residues of the amino acid sequence shown in SEQ ID NO:2 and encompasses an epitope of XBP1 such that an antibody raised against the peptide forms a specific immune complex with the XBP1 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of XBP1 that are located on the surface of the protein, e.g., hydrophilic regions, as well as regions with high antigenicity.

A XBP1 immunogen is typically used to prepare antibodies by immunizing a suitable subject, (e.g., rabbit, goat, mouse, or other mammal) with the immunogen. An appropriate immunogenic preparation can contain, for example, recombinantly expressed XBP1 protein or a chemically synthesized XBP1 polypeptide. The preparation can further include an adjuvant, such as Freund's complete or incomplete adjuvant, or similar immunostimulatory agent. Immunization of a suitable subject with an immunogenic XBP1 preparation induces a polyclonal anti-XBP1 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a XBP1. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind XBP1 molecules. The term “monoclonal antibody” or “monoclonal antibody composition”, as used herein, refers to a population of antibody molecules that contain only one species of an antigen binding site capable of immunoreacting with a particular epitope of XBP1. A monoclonal antibody composition thus typically displays a single binding affinity for a particular XBP1 protein with which it immunoreacts.

Polyclonal anti-XBP1 antibodies can be prepared as described above by immunizing a suitable subject with a XBP1 immunogen. The anti-XBP1 antibody titer in the immunized subject can be monitored over time by standard techniques, such as with an enzyme linked immunosorbent assay (ELISA) using immobilized XBP1. If desired, the antibody molecules directed against XBP1 can be isolated from the mammal (e.g., from the blood) and further purified by well-known techniques, such as protein A chromatography to obtain the IgG fraction. At an appropriate time after immunization, e.g., when the anti-XBP1 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somat. Cell Genet. 3:231-36). Briefly, an immortal cell line (typically a myeloma) is fused to lymphocytes (typically splenocytes) from a mammal immunized with a XBP1 immunogen as described above, and the culture supernatants of the resulting hybridoma cells are screened to identify a hybridoma producing a monoclonal antibody that binds XBP1.

Any of the many well known protocols used for fusing lymphocytes and immortalized cell lines can be applied for the purpose of generating an anti-XBP1 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind XBP1, e.g., using a standard ELISA assay.

Alternative to preparing monoclonal antibody-secreting hybridomas, a monoclonal anti-XBP1 antibody can be identified and isolated by screening a recombinant combinatorial immunoglobulin library (e.g., an antibody phage display library) with XBP1 to thereby isolate immunoglobulin library members that bind XBP1. Kits for generating and screening phage display libraries are commercially available (e.g., the Pharmacia Recombinant Phage Antibody System, Catalog No. 27-9400-01; and the Stratagene SurfZAP™. Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-XBP1 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-XBP1 antibody can be used to detect XBP1 protein (e.g., in a cellular lysate or cell supernatant) in order to evaluate the abundance and pattern of expression of the XBP1 protein. Anti-XBP1 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figures and the Sequence Listing, are incorporated herein by reference.

### SPECIFIC EXAMPLES

### Example 1

### Complementary Signaling Pathways Regulate the Unfolded protein Response and are Required for Development

### A. Materials and Methods

### Stains and General Methods

The strain N2 (Bristol) was used as the wild-type strain. The strain JJ529 rol-1(e91) mex-1(zu121)/mnC1 [dpy-10(e128) unc-52(e444)] was used to construct IRE1(v33)/mnC1; PEK1(ok275) (Sigurdson et al., 1984). C. elegans strains were cultivated at 20° C. unless otherwise indicated (Brenner, 1974).

### Drug Treatments

For Northern analysis, mixed-stage nematodes grown in liquid culture were treated with 3 mM of DTT (Calbiochem) for up to 8 hours. Heat-shock treatment was performed at 30° C. for 1 hour. For single worm analysis, individual L2 larvae grown on plates were treated with 2.5 mM of DTT or 28 μg/ml of tunicamycin (Calbiochem) for 4 hours. To study survival to tunicamycin, gravid adults were allowed to lay eggs on plates containing tunicamycin (0 to 7.5 μg/ml) for 4 hours and then removed from the plates. Eggs were counted and studied 3 days later.

### Northern Blot Analysis

Total RNA preparation and Northern blot analysis was performed as described (Chen et al., 2000). The blot was hybridized sequentially with digoxigenin (DIG)-labeled DNA probes for hsp-3 and for 26S rRNA prepared with DIG-high prime labeling Kit (Roche).

RT-PCR and DNA Sequence Analysis of xbp-1 Transcripts

RNA was isolated from wild-type L2 larvae and mixed-staged IRE1 mutants treated with DTT or tunicamycin (MRC, Inc). First-strand cDNA was synthesized using oligo-dT primer (Promega) and amplified using primers T7-R743F and R743-2R. PCR fragments were sequenced. PCR-amplified first-strand cDNA with the primers T7-R743F and R743-3R, producing a 426 bp (unspliced) and a 403 bp fragment (spliced), that were separated on a 2.2% agarose gel.

In vitro RNA Cleavage

In vitro cleavage of xbp-1 mRNA was performed as described by Sidrauski et al. (1997) and Tirasophon et al. (1998). A 399 bp wild-type xbp-1 DNA fragment fused to the T7 promoter was amplified from C. elegans first strand cDNA using the primers (T7-R743F and R743-3R). T7 promoter fused mutant xbp-1 DNA fragments were generated by overlapping PCR using primer pairs: CE-5′G(−1)C/-AS, CE-5′A(−2)T/-AS, CE-5′C(−3)G/-AS, CE-5′G(+3)C/-AS, CE-3′G(−1)C/-AS, CE-3′A(−2)T/-AS, CE-3′C(−3)G/-AS, and CE-3′G(+3)C/-AS. The [³²P]-labeled xbp-1 RNA was produced by in vitro transcription (Boehringer Mannheim). Wild-type and endoribonuclease mutant (K907A) hIRE1α were prepared as described (Tirasophon et al., 1998). Purified xbp-1 RNA fragments were incubated with wild-type or mutant hIRE1α proteins at 30° C. for 1 hour. The reaction mixes were separated on 5% denaturing polyacrylamide gels, and analyzed by autoradiography.

### Quantitative Taqman RT-PCR Analysis

Taqman RT-PCR was performed as described by Heid et al. (1996). The lack of DNA contamination in RNA preparations was confirmed by a 1000-fold decrease in quantitative PCR yield when reverse transcriptase was omitted. A unique sequence in the 3′UTR of act-1 and act-3 was amplified using Taqman PCR (primers act-F/act-R and act-probe). The primers (hsp-3-F/hsp-3-R) and the hsp-3-probe probe were used for detecting hsp-3 transcripts. The primers (hsp-4-F and hsp-4-R) and the hsp-4-probe probe were used for detecting hsp-4 transcripts. The relative expression of hsp-3 and hsp-4 was normalized to the average signals of act-1/act-3.

### RNA Interference

PCR was used to amplify fragments flanked by the T7 promoter at both the 5′ and 3′ ends. The primer pair T7IF and T7IR amplified a 521 bp region from the ATG start codon on the IRE1 gene. T7PF and T7PR amplified a 526 bp region from the ATG start codon on the PEK1 gene. T7-R743F and T7-R743R amplified a 480 bp of exon II of the xbp-1 gene. Amplified templates were transcribed in vitro to yield dsRNA (Chen et al., 2000) for injection as described (Fire et al., 1998). Only progeny hatched from eggs laid between 12 to 24 hours post-injection were studied.

Isolation of IRE1(v33) and PEK1(ok275) Deletion Mutants

Nested PCR (primers IOF/IOR and IIF/IIR) was used to screen for shorter alleles of IRE1 in an EMS mutagenized worm library that was composed of 1.2×10⁶ mutagenized genomes. The wild-type IRE1 allele amplified a 2441-bp fragment compared to a 1564-bp fragment from the IRE1(v33) deletion allele. A homozygous mutant [IRE1(v33)] was identified by PCR using primers T7IF/T7IR from inside the deleted region. The IRE1(v33) mutant strain was back-crossed five times to animals of N2 background. Nested PCR (POF/POR and PIF/PIR) was used to characterize the PEK1(ok275) deletion mutant, which generated a 952-bp fragment compared to a 2965 by fragment from the wild-type allele. A PCR reaction with the primers PF5 (from inside the deletion region) and PR2 identified homozygous PEK1 mutants.

Construction of the Strain IRE1(v33)/mnC1; PEK1(ok275)

First, PEK1(ok275) males were mated to the rol-1(e91) mex-1(zu121)/mnC1 [dpy-10(e128) unc-52(e444)] hermaphrodites. The mnC1/+; PEK1(ok275)/+hermaphrodite progeny were mated to IRE1(v33)/+males. Then, IRE1(v33)/mnC1; PEK1(ok275)/+animals were selected by the PCR, and self-fertilized to generate the IRE1(v33)/mnC1; PEK1(ok275) strain.

### B. Results

Transcription of hsp-3 and hsp-4 is Induced upon ER Stress in C. elegans.

The most well characterized transcriptional target of the UPR is the gene encoding BiP (grp78) (Kaufman, 1999). C. elegans has two homologues of mammalian BiP, HSP-3, with a KDEL ER-retention motif, and HSP-4, with a HDEL ER-retention motif. By contrast, yeast and mammals have either BiP-HDEL or BiP-KDEL, respectively (Kaufman, 1999). In order to determine whether C. elegans has a UPR, the expression of hsp-3 and hsp-4 were analyzed upon ER stress induced by dithiothreitol (DTT), a reducing reagent that disrupts disulfide bond formation in the ER. Northern blot and quantitative Taqman RT-PCR analysis showed that in mixed-stage worms grown in liquid culture, expression of both hsp-3 and hsp-4 increased with time, and reached a plateau at 4 hours (FIGS. 5A and 5B). At the plateau, hsp-3 was induced about 2-fold, and hsp-4 was induced about 9-fold in mixed-stage animals. Furthermore, the basal expression of hsp-3 was about 5-fold higher than hsp-4. Potential UPR regulatory elements in the promoters of hsp-3 and hsp-4 are set forth in FIG. 5C. Thus, C. elegans has a UPR and Taqman RT-PCR allows us to analyze the UPR in single worms.

RNA Interference Shows that Either IRE1 or PEK1 is Required for Larval Development in C. elegans.

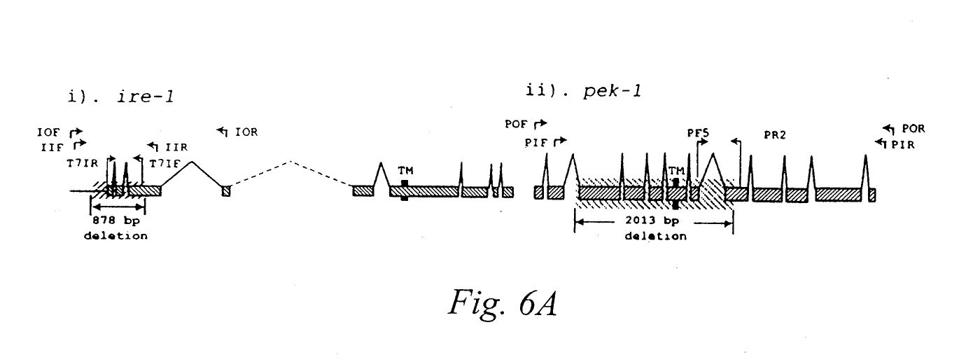

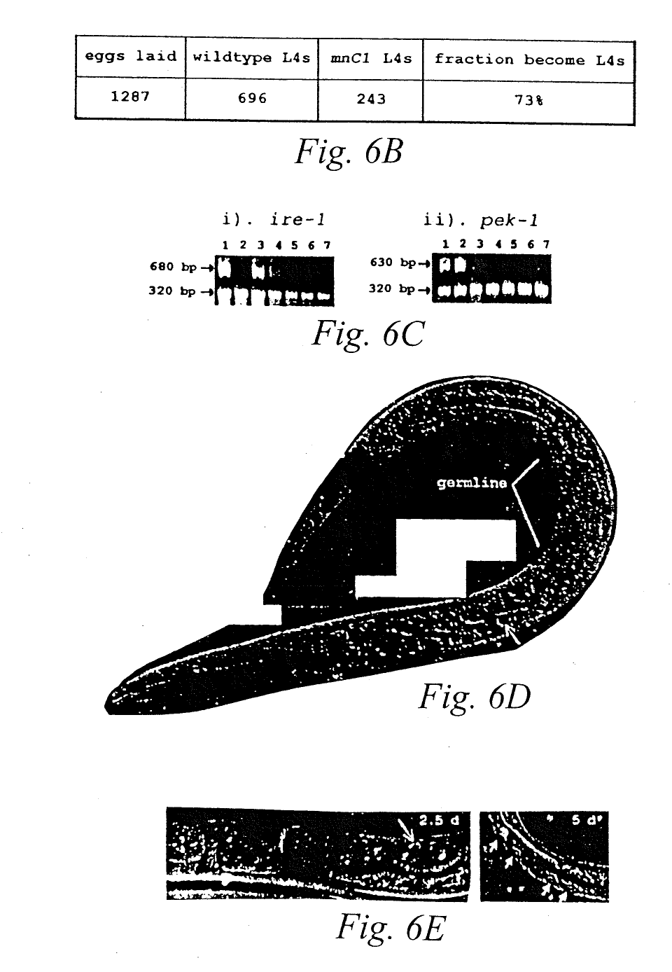

The C. elegans homologues for mammalian IRE1 and Perk were designated as IRE1 and PEK1, respectively. Using RT-PCR, both genes were cloned and sequenced (FIG. 6A); Genebank accession number: AF435952 for IRE1 and AF435953 for PEK1). To study the requirements for IRE1 and PEK1 in the UPR and development, RNA interference (RNAi) was used to inactivate each gene. The IRE1(RNAi) and PEK1(RNAi) animals displayed a linear growth identical to the mock control—progeny from adults injected with buffer alone. They became late L2 larvae at 1.5 days after eggs were laid and matured to adulthood at 3 days. The IRE1(RNAi); PEK1(RNAi) animals also became early L2 larvae by 1.5 days, and at that time were indistinguishable from controls or the single mutants. However, after the IRE1(RNAi); PEK1(RNAi) animals reached L2, they became very sluggish and sick. Six days after eggs were laid 90% (n=120) remained as L2 larvae, identified by germline morphology.

Close examination of IRE1(RNAi); PEK1(RNAi) animals revealed small vacuoles in the intestinal cells at 1.5 days after the eggs were laid. These vacuoles increased in number and size by 2.5 days (FIG. 6E). By 4 days, the connection between the intestine and pharynx narrowed, so bacteria could not pass through to the intestine. Furthermore, the intestine fragmented, and large empty spaces appeared in the worm. By 5 or 6 days, most intestinal tissues degraded and the cytoplasm of the intestinal cells disappeared, with only nuclei remaining distinct (FIG. 6E). This phenotype is characteristic of necrosis (Wyllie et al., 1981; Hall et al., 1997). These RNAi results show that IRE1 and PEK1 are redundant genes that control a pathway essential for larval development.

### IRE1 and PEK1 Deletion Mutants are Viable.

To confirm the RNAi results, deletion mutants of IRE1 and PEK1 were identified. The IRE1(v33) null mutation was isolated from an EMS-mutagenized worm library by screening short alleles by nested PCR. An 878-bp deletion was found extending from −199 bp upstream of the ATG start codon to by 679 of the IRE1 gene (FIG. 6A). The IRE1(v33) mutants were viable, but their growth was somewhat slower than observed for wild-type animals.

The PEK1(ok275) mutant (isolated by the C. elegans Gene Knockout Consortium, Oklahoma) had a 2013-bp deletion, extending from 495 bp to 2507 bp in the PEK1 gene. Sequencing analysis showed that the transcript was missing a 1535-bp (from 280 bp to 1815 bp) fragment that included exons 3 to part of exon 8 (FIG. 6A). Although the deletion was in frame, loss of the transmembrane domain predicts the mutant PEK1 is mislocalized to the ER lumen, causing a loss-of-function. These PEK1(ok275) mutants were indistinguishable from the wild-type under normal growth conditions.

IRE1(v33); PEK1(ok275) Double Mutants Arrest as L2 Larvae with Intestinal Degeneration

To ensure a stable supply of the IRE1(v33); PEK1(ok275) homozygous mutants, strain IRE1(v33)/mnC1; PEK1(ok275) were constructed, in which the IRE1(v33) mutation is balanced by the marker chromosome mnC1. According to Mendelian genetics, one-quarter of the progeny should be IRE1(v33); PEK1(ok275) homozygotes. A total of 1287 eggs were laid by IRE1(v33)/mnC1; PEK1(ok275) animals. Three days after eggs were laid, 27% of IRE1(v33)/mnC1; PEK1(ok275) progeny failed to mature into wild-type (the phenotype of the heterozygous parents) or Dyp Unc adults (the phenotype of mnC1) (FIG. 6B). Instead, many L2-arrested animals were observed having IRE1(v33); PEK1(ok275) genotypes (FIG. 6C). These IRE1(v33); PEK1(ok275) double mutants showed intestinal degeneration like that observed in the RNAi studies (FIG. 6D).

xbp-1 mRNA is an IRE1 Substrate Required for IRE1 Signaling

To elucidate the mechanism for hsp-3 and hsp-4 induction, their promoter regions were analyzed. The hsp-3 promoter has three ERSE-I-like sequences (ER stress element, FIG. 5C) (Yoshida et al., 1998). In contrast, hsp-4 lacks ERSE-I sites but has two identical sequences similar to ERSE-II (Kokame et al., 2000). In addition, the hsp-4 promoter contains a mammalian XBP1 (X-box DNA binding protein) binding site (Clauss et al., 1996), while the hsp-3 promoter contains three ATF/CREB recognition sites (Kataoka et al., 1994; Koldin et al., 1995). The mammalian XBP1 recognition site in the hsp-4 promoter suggested the potential importance of C. elegans XBP-1 in regulation of hsp-4 expression upon ER stress.

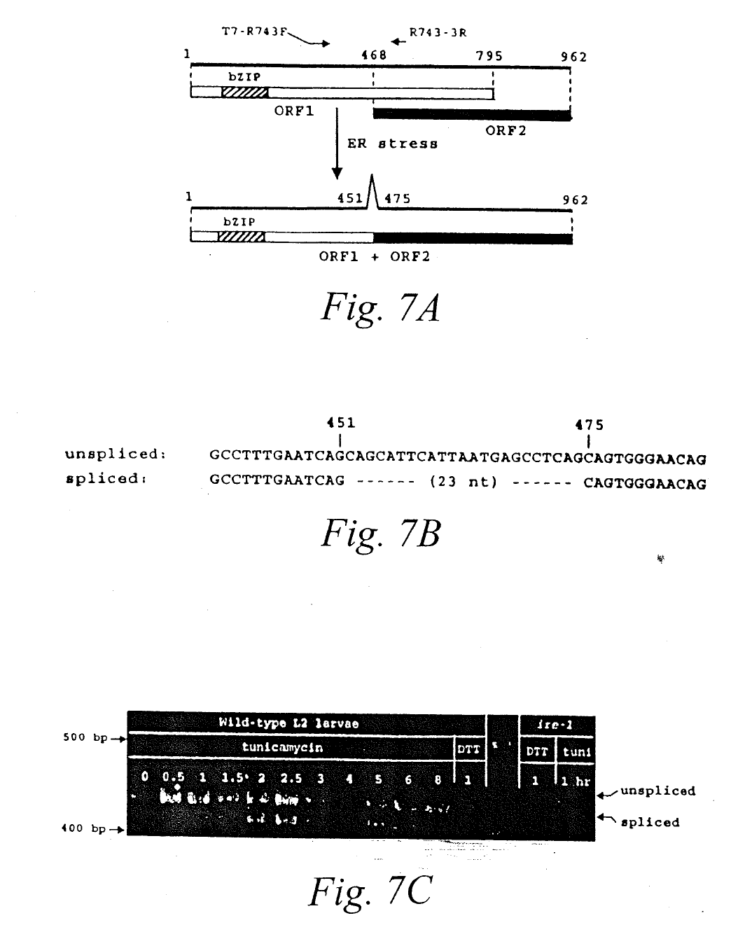

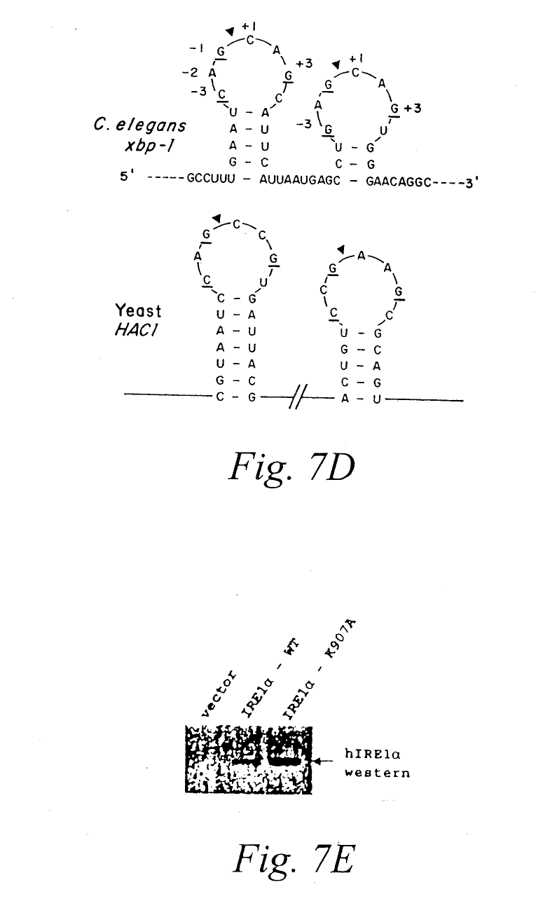

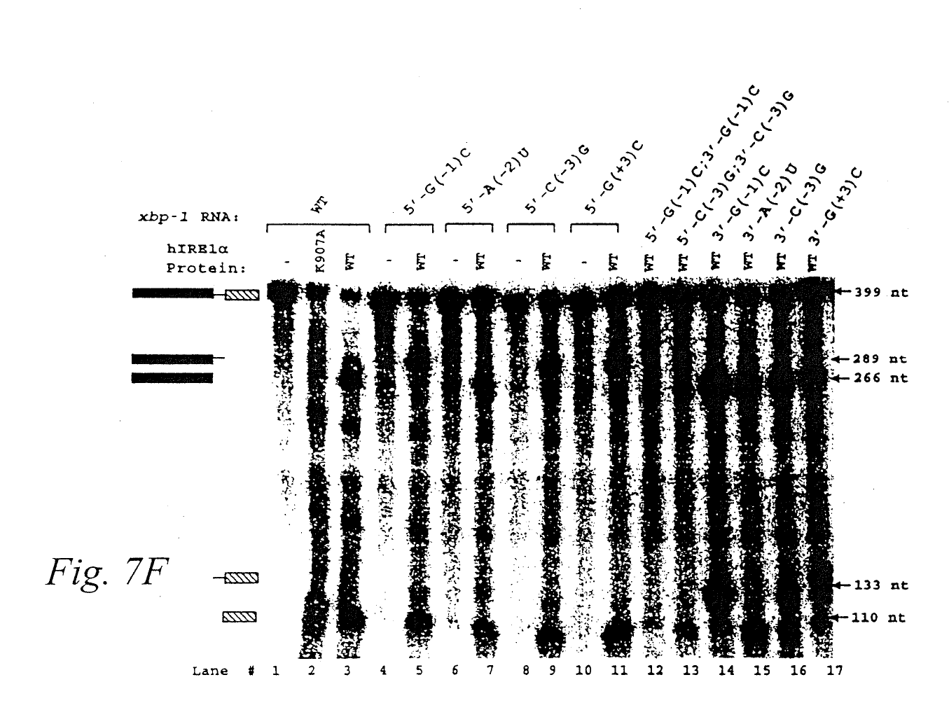

In the course of these studies, a putative mammalian homologue was identified for yeast HAC1—the bZIP transcription factor Xbp1 (Yoshida et al., 2001b). The protein sequence of human XBP1 was used to search the C. elegans protein database, and a hypothetical protein encoded by the gene R74.3 was identified, which we designated xbp-1. C. elegans XBP-1 has a conserved bZIP domain and shares no amino acid homology with human XBP1 or yeast HAC1 outside of the bZIP region. The xbp-1 gene contains an additional open reading frame that is in +1 register with the xbp-1 initiation AUG codon (FIG. 7A). Quantitative Taqman RT-PCR showed that total xbp-1 transcription increased 2˜3 fold upon ER stress induced by DTT or by inhibition of N-linked glycosylation by tunicamycin treatment (data not shown). Splicing of xbp-1 mRNA to remove 23 bases was induced between 30 min-1 h after tunicamycin treatment in wild-type L2 larvae (FIGS. 7B and 7C). Significantly, this novel mRNA species was not detected in IRE1(v33) mutants (FIG. 7C). Therefore, excision of the 23 base sequence requires IRE1 and would generate a +1 translation shift into the second reading frame.

The 23 base intron is predicted to form an RNA secondary structure containing two stem-loop signatures with seven-membered rings, similar to that found in yeast HAC1 (FIG. 7D). To test whether xbp-1 mRNA can be cleaved by IRE1, an in vitro cleavage assay was performed using human IRE1α expressed in COS-1 monkey cells. Western blot analysis confirmed that both the wild-type and endoribonuclease mutant (K907A) IRE1α were expressed (FIG. 7E). Human IRE1α cleaved the C. elegans xbp-1 RNA substrate (399 nt fragment) at the expected 5′ and 3′ cleavage sites, releasing the 23 nt intron and yielding two fragments (266 nt and 110 nt) that were detected on a polyacrylamide gel (FIG. 7F, lane 3). Although the IRE1α endoribonuclease mutant (K907A) was expressed at a much higher level as previously described (Tirasophon et al., 1998 and 2000), it cleaved xbp-1 RNA to a much lesser extent (FIG. 7F, lane 2). These results demonstrate that the RNase activity of IRE1α is required for xbp-1 cleavage. Mutation of the conserved sites (−3, −1, and +3) in both the 5′ and 3′ loops interfered with the cleavage reaction (FIG. 7F, lanes 5, 9, 11, 14, 16, and 17). By contrast, mutation of the nonconserved base (−2) in either the 5′ or 3′ loop did not prevent cleavage (FIG. 7F, lanes 7 and 15). Moreover, double mutations at either −1 or −3 sites within both the 5′ and 3′ loops abolished or significantly reduced cleavage, respectively (FIG. 7F, lanes 12 and 13).

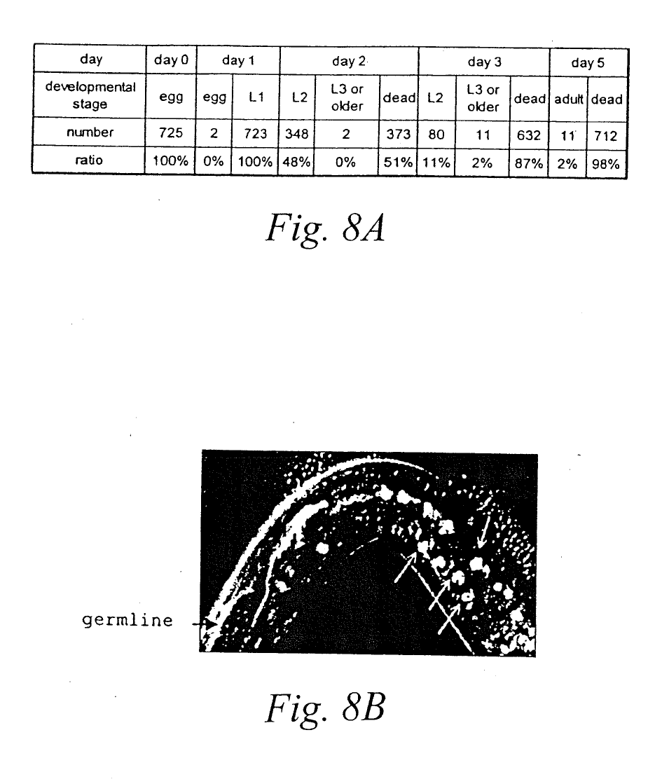

The genetic interaction between xbp-1 and PEK1 was also tested. Though PEK1(ok275); xbp-1(RNAi) eggs hatched normally, they arrested at or prior to the L2 larval stage (FIG. 8A). In addition, the PEK1(ok275); xbp-1(RNAi) animals showed an intestinal defect resembling that of IRE1(v33); PEK1(ok275) double mutants (FIG. 8B). By contrast, inactivating xbp-1 in either IRE1 or in wild-type worms did not interfere with development. Therefore, RNAi experiments demonstrated that xbp-1 and PEK1 mediate redundant pathways that are essential for worm development, and our results are consistent with xbp-1 acting downstream of IRE1 in the same pathway.

IRE1, xbp-1 and PEK1 are Required for the UPR in C. elegans

To determine if silencing IRE1, xbp-1 and PEK1 expression would affect the UPR, quantitative Taqman RT-PCR was used to analyze hsp-3 and hsp-4 expression in affected animals. Since IRE1(v33); PEK1(ok275) and PEK1(ok275); xbp-1(RNAi) mutants did not grow to adulthood, it was believed that the pathway mediated by IRE1/xbp-1 and PEK1 might be required for L2 development. Therefore, individual 1.5 day-old L2 larvae were studied. Expression of the two hsp genes was normalized to that of act-1 and act-3.

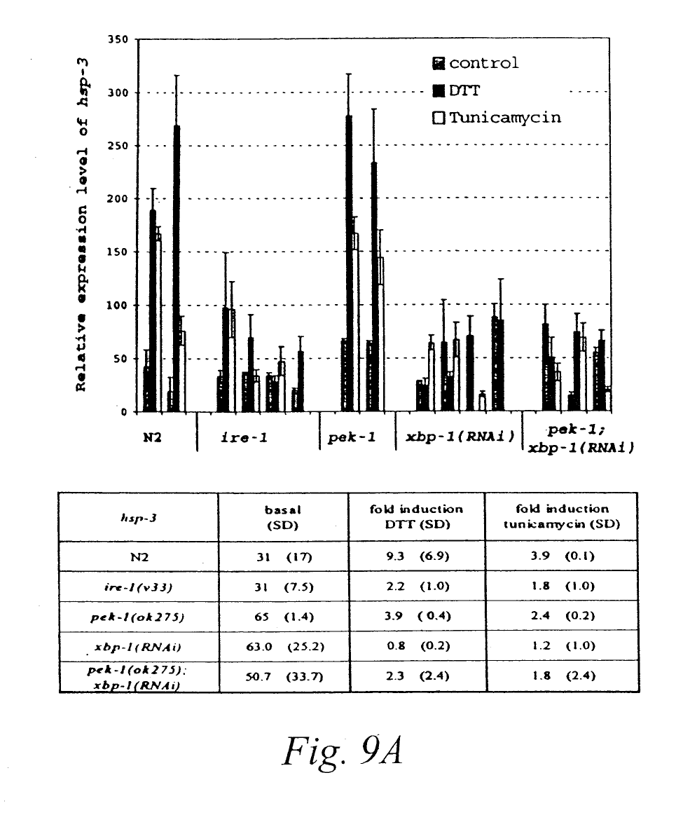

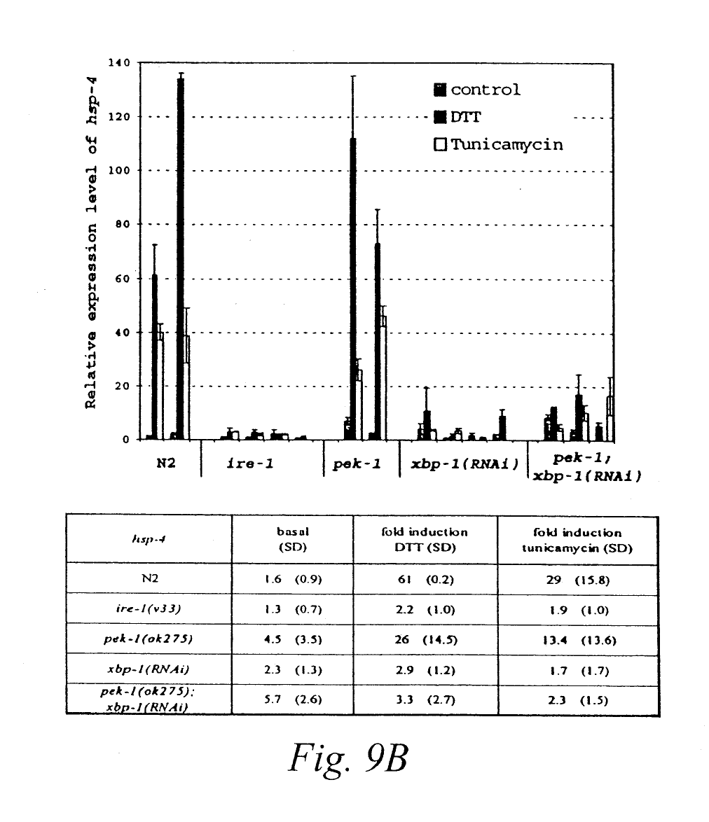

In wild-type L2 larvae, the basal expression of hsp-3 was about 19-fold higher than that of hsp-4 (FIGS. 9A and 9B). In contrast to 2- and 10-fold induction of hsp-3 and hsp-4, respectively, in mixed-staged animals (FIG. 5B), in L2-stage larvae expression of hsp-3 and hsp-4 was induced ˜9.3-fold and 61-fold, respectively, upon DTT treatment. Furthermore, expression of hsp-3 and hsp-4 was induced ˜3.9- and 29-fold, respectively, upon tunicamycin. In IRE1(v33) mutants, the basal expression of the two hsp genes was similar to that of N2 animals. However, the induction of the hsp-3 gene by DTT or tunicamycin was greatly reduced, and that of hsp-4 was almost abolished. Therefore, IRE1 is required to activate the UPR in C. elegans.

As with IRE1(v33) mutants, induction of both hsp genes was abolished in xbp-1(RNAi) animals (FIGS. 9A and 9B). Furthermore, PEK1(ok275); xbp-1(RNAi) animals were defective in inducing both hsp genes. By contrast, PEK1(ok275) mutants were able to activate transcription of both hsp genes to a similar extent as the wild-type. However, the basal expression of both hsp genes was increased in the PEK1(ok275) mutant. It is possible that PEK1(ok275) mutants experience endogenous ER stress during development, consistent with a model where PEK1 limits ER stress by attenuating protein synthesis. Overall, these results suggest that IRE1/xbp-1 and PEK1 play partially complementary roles in eliminating ER stress, where IRE1/xbp-1 signals to activate UPR transcription and PEK1 signals to attenuate protein synthesis.

### Mutant Animals are Sensitive to Tunicamycin

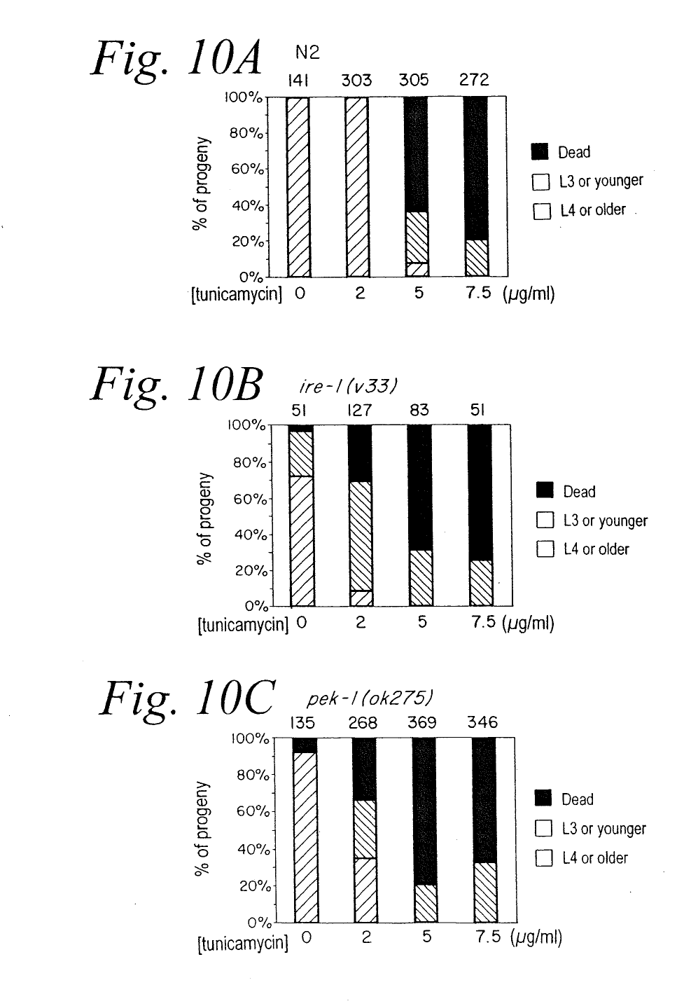

The survival of wild-type (N2) and mutants upon induction of ER stress by tunicamycin was studied. The growth of N2 animals was not affected until the tunicamycin concentration reached 5 μg/ml. At 5 μg/ml, only 8% of N2 matured to the L4 stage or older after 3 days, 29% were arrested at or prior to the L3 stage, and 63% were dead. The arrested N2 animals had many vacuoles in their intestinal cells (data not shown). These vacuoles were indicative of a necrotic cell death, much like that observed in IRE1(v33); PEK1(ok275) mutants. In the absence of tunicamycin, 72% of IRE1(v33) mutants matured to the IA stage or older within 3 days. On plates with 2 μg/ml of tunicamycin, only 9% of IRE1 animals matured to the L4 stage or older, 60% arrested at or prior to the L3 stage and 31% were dead. As for PEK1(ok275) mutants on plates with 2 μg/ml of tunicamycin, only 35% matured to the L4 stage or older, 31% arrested at or prior to the L3 stage and 34% were dead. Thus, both LRE1(v33) and PEK1(ok275) mutants were sensitive to tunicamycin treatment at 2 μg/ml, whereas N2 animals were resistant to this concentration (FIG. 10A). Furthermore, IRE1(v33) mutants appeared more sensitive to tunicamycin than did PEK1(ok275) mutants (FIGS. 10B and 10C). The double mutant was exquisitely sensitive to tunicamycin (data not shown). These results demonstrate that IRE1 and PEK1 provide adaptive functions upon ER stress.

Comparison of the UPR in C. elegans and S. cerevisiae

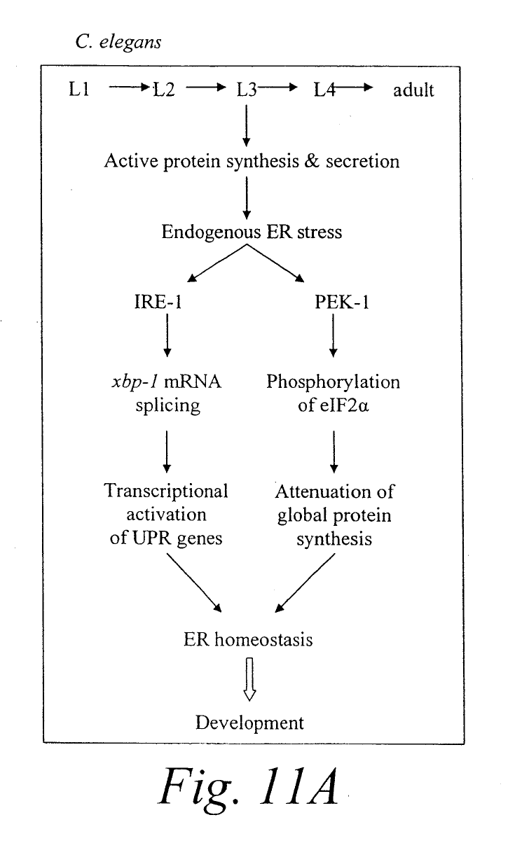

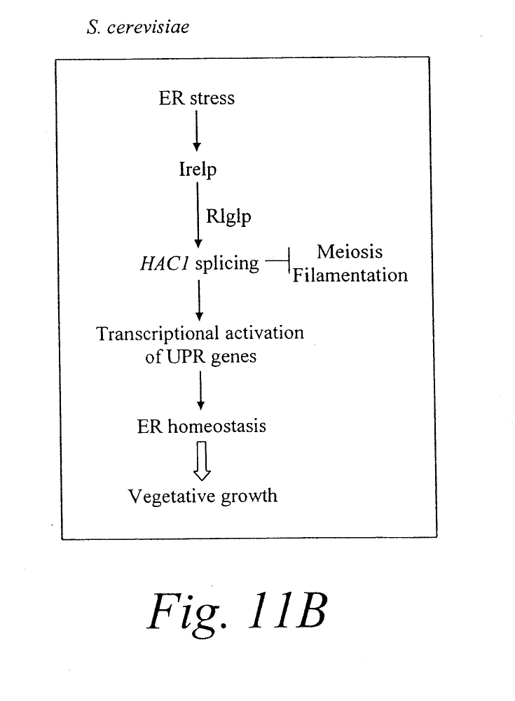

During development, active protein synthesis and secretion might generate endogenous ER stress, which would activate IRE1 and PEK1. Activated IRE1 splices xbp-1 mRNA, resulting in translation of an active bZIP transcriptional factor. The transcriptional activation of the UPR in C. elegans is controlled by IRE1 and XBP-1, and attenuation of global protein synthesis by PEK1, should increase the folding capacity of the cell and decrease the protein-folding load, so that ER homeostasis is maintained, allowing for proper development (FIG. 11A).

In S. cerevisiae, the UPR is a simple, linear pathway requiring only IRE1p, the bZIP transcription factor Hac1p, and tRNA ligase Rlg1p (Sidrauski et al., 1996) (FIG. 11B). ER stress-induced HAC1 mRNA splicing mediated by IRE1p suppresses yeast differentiation and allows vegetative growth (Schroder et al., 2000).

The references cited in Example 1 may be found in Shen, X. et al., Cell 107:893-903 (2001), expressly incorporated by reference herein.

### Example 2

### IRE1-Mediated Unconventionals mRNA Splicing and S2P-Mediated ATF6 Cleavage Merge to Regulate XBP1 in Signaling the Unfolded Protein response

### A. Materials and Methods

### Cell Culture and Transient DNA Transfection

Culture methods and media for COS-1 monkey cells were previously described (Kaufman, 1997) and the same methods were applied to MEFs except that fetal bovine serum (FBS) was not heat-inactivated. Wild-type (K1) and S2P-deficient (clone M19) Chinese hamster ovary (CHO) cells were cultured as described (Ye et al., 2000). R1 murine embryonic stem (ES) cells (Joyner, 1989), were plated onto mitomycin C-treated MEF feeder cells in ES cell medium ((Dulbecco's Modified Eagle Medium (GIBCO BRL, Rockville, Md.)) supplemented with 15% heat-inactivated FBS, 0.1 mM (3-mercaptoethanol and 1000 units/ml Leukocyte Inhibitory Factor (GIBCO BRL, Rockville, Md.). COS-1 cells were transfected by either Diethylaminoethyl(DEAE)-Dextran (Kaufman, 1997) or Calcium-Phosphate-BES methods (Ausubel et al., 1999). MEFs were transfected by either FuGENE6 (Roche, Germany) or Effectine (Qiagen, Germany) according to the manufacture's recommended procedures. CHO cells were transfected by FuGENE6 (Roche, Germany).

### Construction of IRE1α Targeting Vector and Gene Disruption

A XbaI-NotI fragment of a loxP neomycin resistance cassette under control of the phosphoglycerate kinase (PGK) promoter (Orban et al., 1992) was inserted into a murine IRE1α fragment to replace exons 7 to 14 yielding the BS-mIRE1α targeting vector. Trypsinized R1 ES cells were mixed with NotI digested BS-mIRE1α targeting vector and a high electric pulse (250 ° F. and 0.3 kV) was applied using a gene-puller (Bio-Rad laboratories, Hercules, CA). The transfected cells were plated onto MEF feeder cells at a density of 10⁶ cells/100-mm plate. Selection medium containing 300 μg/ml G418 (GIBCO BRL, Rockville, Md.) was applied to the ES cells at 48 hr post-transfection. G418 resistant colonies formed at 4-5 days after selection were isolated for screening.

### RT-PCR and Plasmid Construction

Xbp1 RNA splicing was detected by standard RT-PCR using total RNA templates isolated from MEFs treated with or without tunicamycin (10 g/ml, 6 hr) using oligo d(T)₁₅ and specific primers; mXbp1-354 (5′ ccttgtggttgagaaccagg 3′ (SEQ ID NO:5)) and mXbp1-804-AS (5′ ctagaggcttggtgtatac 3′ (SEQ ID NO:6)). The spliced form of Xbp1 cDNA was obtained by RT-PCR using RNA templates obtained from MEFs treated with tunicamycin and oligo d(T)₁₅, mXbp1-354, and mXbp1-1150-R1 (5′ cgaattcttagacactaatcagc 3′ (SEQ ID NO:7)) as primers. The spliced form of Xbp1 cDNA, pcDNA3-Xbp1-s, was constructed by subcloning the 0.7-kb BamHI-EcoRI RT-PCR to fragment from Xbp1 into the respective sites in pcDNA3-Xbp1-ORF1. The unspliced form of full-length Xbp1, pcDNA3-Xbp1-u, was constructed using RNA templates obtained from IRE1α-null MEFs without tunicamycin treatment. DNA sequence analysis was performed to verify PCR-amplified DNA sequences.

### Pulse-Chase Analysis of ATF6

Wild-type and IRE1α-null MEFs cultured on 100-mm plates were pulse-labeled with [³⁵S]-methionine and [³⁵S]-cysteine (0.5 mCi/100-mm dish, 1000 Ci/mmole, Amersham Pharmacia, Piscataway, N.J.) for 40 min and then chase performed with or without 10 μg/ml tunicamycin for the times indicated. Proteins were extracted and immunoprecipitated using anti-ATF6 antibody as previously described (Haze et al., 1999) and subjected to SDS-PAGE (10% gel). Radiolabeled proteins were analyzed using a Phospholmager (Molecular Dynamics).

### 5× ATF6, BiP, and GAL4 Reporter Assays

The reporter plasmids containing the luciferase gene under control of five ATF6 binding sites or the GAL4 DNA binding site (Wang et al., 2000) and the BiP promoter (Tirasophon et al., 1998) were previously described. Reporter assays was performed as previously described (Tirasophon et al., 2000) with an exception that a plasmid containing β-galactosidase under control of the CMV promoter was used to correct for transfection efficiency.

### Southern and Northern Blot Analysis

Southern and Northern blot analysis followed standard procedures (Sambrook et al., 1989). [³²P]-labeled probes were prepared using a random prime labeling system (Amersham Pharmacia, Piscataway, N.J.). A 0.5-kb BamHI-XhoI fragment from the BS-mIRE1α targeting vector or a 3.6-kb EcoRI-XbaI fragment from pED-hIRE1α cDNA (Tirasophon et al., 2000) were used for Southern and Northern analysis, respectively. The probes for Northern analysis of mXbp1, BiP and GRP94 were a 0.94-kb XhoI fragment of pcDNA-mXbp1-u, the EcoRI-PstI fragment of hamster BiP (Ting et al., 1987), and a 146-bp PCR fragment of mouse GRP94 (from 142 to 287 of the coding region), respectively.

### Immunoprecipitation and Western Blot Analysis

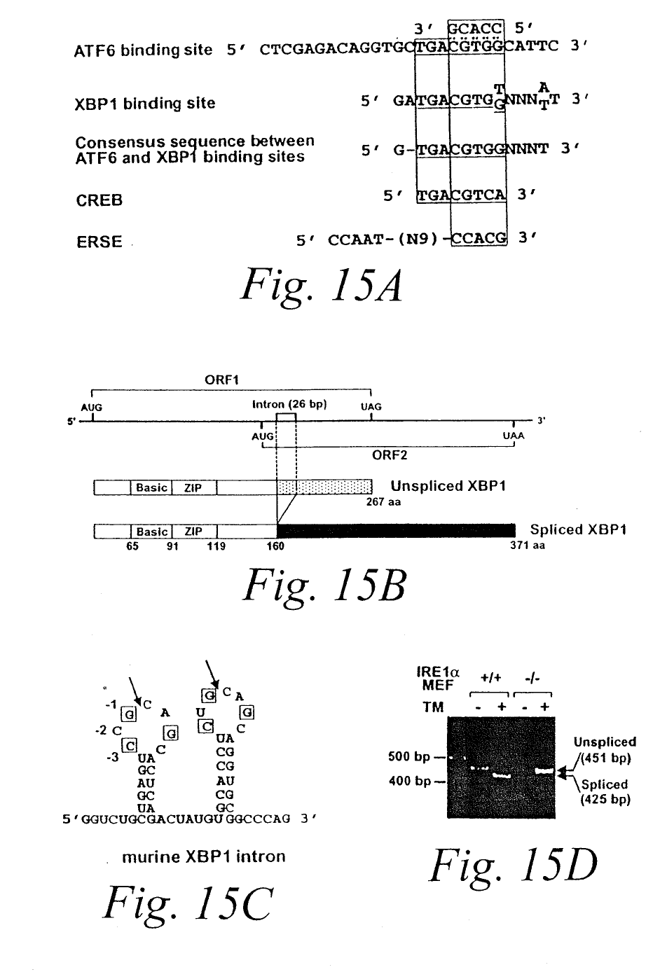

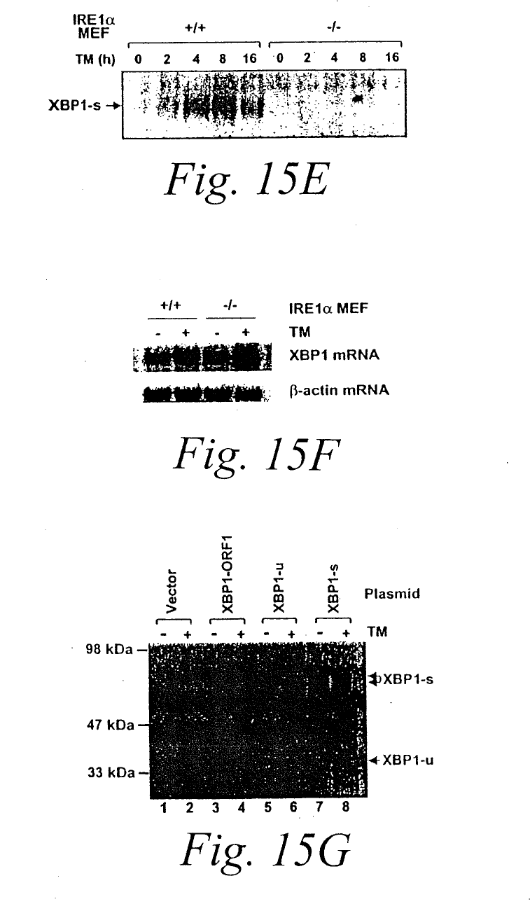

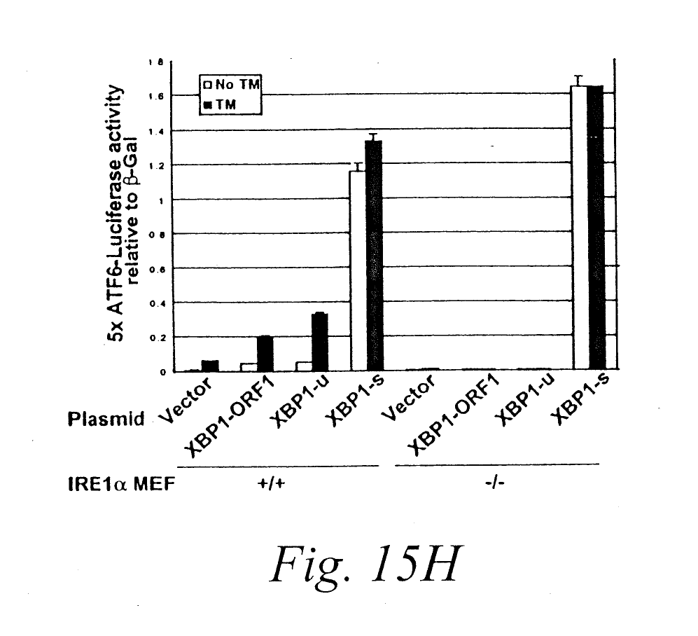

For analysis of ATF6, cells were directly harvested in SDS sample buffer lacking DTT and subjected to Western blot analysis or immunoprecipitation as previously described (Haze et al., 1999). ATF6 proteins were detected using purified anti-ATF6 antibody and anti-rabbit immunoglobulin conjugated with horseradish peroxidase (Amersham Pharmacia, Piscataway, N.J.). For analysis of IRE1α, total cell extracts were prepared from MEFs, a pancretic cell line HIT-T15, or transfected COS-1 cells using Nonidet P-40 lysis buffer (1% NP-40, 50 mM Tris-HCl, pH 7.5, 150 mM NaCl, 0.05% SDS) supplemented with protease inhibitors (Complete Mini, Roche, Germany), 0.1 mM sodium vanadate and 1 mM sodium fluoride. Western blot analysis of IRE1α using anti-hIRE1α-lumenal domain antibody and immunoprecipitation of T7-tagged IRE1α proteins using anti-T7 antibody were previously described (Tirosophon et al., 2000). XBP1 proteins were detected using anti-XBP1-s antibody (FIG. 15E) (Yoshida et al., 2001b) or purified rabbit anti-XBP1 antibody (FIG. 15G).

In vitro Cleavage of XBP1 RNA

In vitro cleavage of murine Xbp1 mRNA was performed as previously described by Sidrauski and Walter (1997). Briefly, a 404-bp BamHI and EcoRl fragment of Xbp1 DNA fragment that contains the intron was amplified by PCR and subcloned into the same sites of pSPT19 (Roche, Germany) which contains the T7 RNA polymerase promoter. Because of polylinker sites between the T7 promoter and 5′ end of the Xbp1 fragment, a 445 base-long transcribed RNA fragment is expected. Mutant Xbp1 DNA fragments were created by overlapping PCR using mutant oligos: mXbp1-5′G−-1)C, 5′ tctgctgagtccccagcac 3′ (SEQ ID NO:8); mXbp1-5′G(−1)C-AS, 5′ gtgctggggactcagcaga 3′ (SEQ ID NO:9); mXbp1-5′C(−2)G, 5′ tctgctgagtcggcagcac 3′ (SEQ ID NO:10); mXbp1-5′C(−2)G-AS, 5′ gtgctgccgactcagcaga 3′ (SEQ ID NO:11); mXbp1-5′G(+3)C, 5′ gtccgcaccactcagactat 3′ (SEQ ID NO:12); mXbp1-5′G(+3)C-AS, 5′ atagtctgagtggtgcggac 3′ (SEQ ID NO:13); mXBP1-3′G(−1)C, 5′ atgtgcacctctccagcag 3′ (SEQ ID NO:14); mXbp1-3′G(−1)C-AS, 5′ ctgctggagaggtgcacat 3′ (SEQ ID NO:15); mXbp1-3′T(−2)A, 5′ atgtgcacctcagcagcag 3′ (SEQ ID NO:16); mXbp1-3′T(−2)A-AS, 5′ ctgctgctgaggtgcacat 3′ (SEQ ID NO:17); mXbp1-3′C(−3)G, 5′ atgtgcacctgtgcagcag 3′ (SEQ ID NO:18); mXbp1-3′C(−3)G-AS, 5′ ctgctgcacaggtgcacat 3′ (SEQ ID NO:19); mXbp1-3′G(+3)C, 5′ ctctgcaccaggtgcaggc 3′ (SEQ ID NO:20); mXbp1-3′G(+3)C-AS, 5′ gcctgcacctggtgcagag 3′ (SEQ ID NO:21). Xbp1 RNA was transcribed in vitro using T7 RNA polymerase (Roche, Germany) in the presence of [³²P]-UTP (3000 Ci/mmole, Amersham Pharmacia, Piscataway, N.J.). The [³²P]-labeled Xbp1 RNA was purified by electrophoresis in a 5% denaturing polyacrylamide gel, eluted, precipitated and dissolved in endoribonuclease buffer (20 mM Hepes pH 7.3, 1 mM dithiothreitol, 10 mM magnesium acetate, 50 mM potassium acetate, 2 mM ATP). Purified RNA (3×10⁴ cpm) was added to the immunoprecipitated wild-type and endoribonuclease mutant K907A hIRE1α which contain T7-epitope tags at their C-termini (Tirasophon et al., 2000) and incubated at 30° C. for 1 hr. The reactions were terminated by extraction with phenol/chloroform, precipitated with ethanol, and analyzed by electrophoresis on 5% denaturing polyacrylamide gels. Gels were dried prior to autoradiography.

### Isolation and Extraction of Nuclei

Nuclei were isolated from MEFs as described (Blobel and Potter, 1966). Cells were homogenized in two volumes of a solution containing 250 mM sucrose, 25 mM KCl, 5 mM MgCl₂ and 50 mM Tris, pH 7.5. The homogenate was over-layed on a step sucrose gradient consisting of 1.62 M and 2.3 M sucrose followed by centrifugation at 124,000×g for 30 min. using a Beckman SW50.1 rotor. The white pellet containing pure nuclei was collected, suspended in a solution containing 25 mM KCl, 5 mM MgCl₂ and 50 mM Tris, pH 7.5 and centrifuged for 12 min at 13,000×g. The pellet contained pure, intact nuclei. To remove the outer nuclear membranes, the purified nuclei were solubilized with 5% Triton X-100 in 25 mM KCl, 5 mM MgCl₂ and 50 mM Tris, pH 7.5 followed by centrifugation at 800×g for 5 min. The supernatant (Triton X-100 soluble fraction) contained solubilized outer nuclear membrane. The final pellet, containing the outer membrane-stripped nuclei, was suspended in a solution containing 25 mM KCl, 5 mM MgCl₂ and 50 mM Tris, pH 7.5 and centrifuged at 13,000×g for 10 mM. Quality of the isolated nuclei was monitored by electron microscopy (Blobel and Potter, 1966).

### B. Results

IRE1α-Null Murine Embryonic Fibroblasts (MEFs) have an Intact UPR

The role for IRE1α in the UPR was studied using IRE1α-null MEFs. Exon 7 to exon 14 from the IRE1α gene was deleted by homologous recombination in R1 embryonic stem cells using a PGK-neo targeting vector (FIG. 12A) and the presence of the deleted IRE1α locus was demonstrated by Southern blot analysis (FIG. 12B). The IRE1α-deletion was confirmed by Northern blot and Western blot analysis. As expected from the homologous replacement, the homozygous IRE1α-null MEFs express a smaller IRE1α mRNA transcript compared to that detected in wild-type MEFs (FIG. 12C). The predicted protein product from the deleted IRE1α allele would lack the ER transmembrane domain so it would likely be mislocalized to the lumen of the ER. Because the endogenous level of IRE1α expression is very low, expression of IRE1α protein was analyzed by immunoprecipitation using an anti-IRE1α lumenal domain antibody and Western blot analysis using the same antibody. As a positive control, IRE1α was analyzed in a tunicamycin-treated pancreatic β-cell line known to express IRE1α. Tunicamycin inhibits N-linked glycosylation and activates the UPR. Under these conditions, only the phosphorylated form of IRE1α protein is detected, as previously described (FIG. 12D, lane 3) (Tirasophon et al., 1998, 2000). Where nonphosphorylated and phosphorylated species of IRE1α were detected in the wild-type MEFs, anti-IRE 1 a antibody-reactive protein was not detected in IRE1α-null MEFs (FIG. 12D, lanes 1 and 2).

To test the requirement for IRE1α in UPR-transcriptional induction, wild-type and IRE1α-null MEFs were treated with tunicamycin for 6 hr and RNA was prepared for Northern blot analysis. Both wild-type and heterozygous IRE1α+/−cells showed comparable BiP mRNA induction upon tunicamycin treatment. However, BiP mRNA induction was also observed in homozygous IRE1α-null MEFs, although quantification of the results suggested a slight reduced induction (10%) in the IRE1α-null MEFs (FIG. 12E). Induction of GRP94 (FIG. 12F) and CHOP-10 (data not shown) mRNAs were also comparable in the wild-type and IRE1α-null MEFs. To determine whether the increase in BiP mRNA observed reflected transcriptional activity of the BiP promoter, the induction of a BiP promoter-luciferase reporter plasmid was studied. Tunicamycin treatment induced luciferase expression from the BiP promoter to similar degrees in wild-type and in IRE1α-null MEFs (FIG. 12G). These results support that Ire1α is not essential for the transcriptional induction of several well-characterized UPR target genes and suggest that at least one additional mechanism for UPR transcriptional induction is intact in IRE1α-null MEFs.

### 5× ATF6 Reporter Activation is Defective in IRE1α-Null MEFs

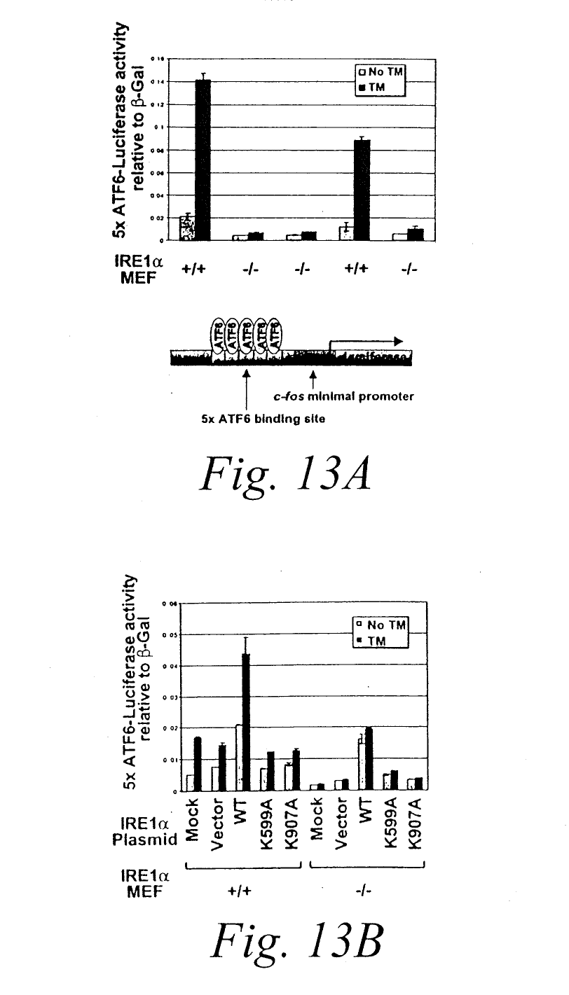

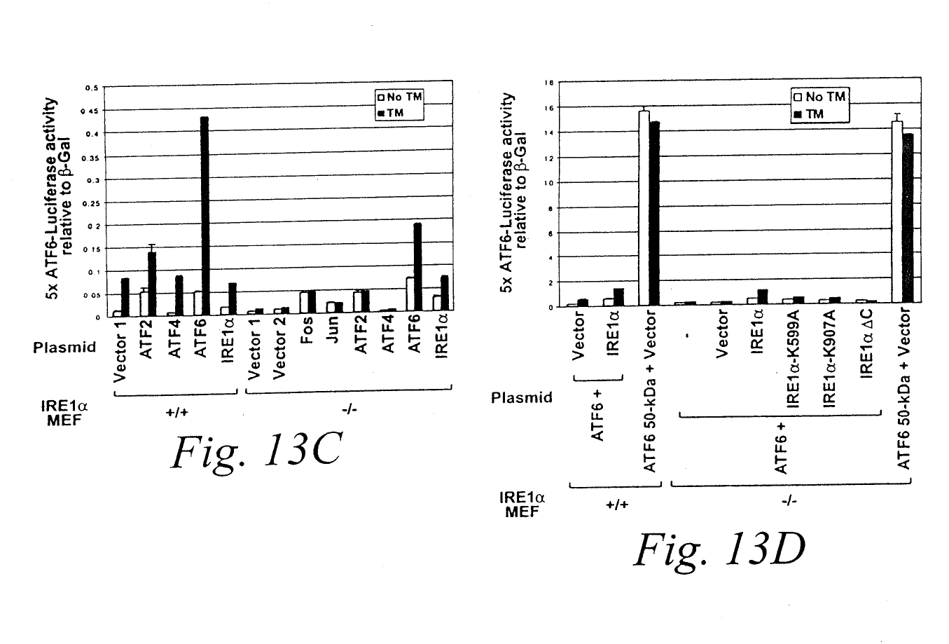

Previous studies support that ATF6 cleavage is required for UPR transcriptional induction (Ye et al., 2000). To test whether IRE1α is required for ATF6 cleavage and function, a luciferase reporter plasmid was used under transcriptional control of a multimerized ATF6 binding site (FIG. 13A, bottom). This multimerized ATF6 binding site is sufficient to direct ER stress-induced expression of luciferase (Wang et al., 2000). Over-expression of wild-type IRE1α activates this 5× ATF6 reporter while over-expression of a kinase and RNase domain-deleted mutant IRE1α (IRE1ΔC) acts in a trans-dominant negative manner to prevent the ER stress-induced expression of the 5× ATF6 reporter (Wang et al., 2000). Surprisingly, compared to wild-type MEFs, tunicamycin-induced expression of the 5× ATF6 reporter gene was completely defective in IRE1α-null MEFs (FIG. 13A). Upon transfection of IRE1α-null MEFs with the 5× ATF6 reporter in the presence of wild-type (WT) IRE1α, kinase-defective K599A mutant IRE1α, or RNase-defective K907A mutant IRE1α, only the wild-type IRE1α complemented the defect in 5× ATF6 reporter expression (FIG. 13B). Therefore, the IRE1α kinase and endoribonuclease activities are required for 5× ATF6 reporter activation. Test were further conducted to determine whether over-expression of several known bZIP/ATF family members could activate 5× ATF6 reporter expression in the IRE1α-null MEFs. Although over-expression of c-Jun, c-Fos and ATF2 slightly increased the basal level of 5× ATF6 reporter gene expression in the IRE1α-null MEFs, no further increase occurred upon tunicamycin treatment. In contrast, over-expression of intact ATF6 elevated both the basal and the tunicamycin-induced 5× ATF6 reporter gene expression in the IRE1α-null MEFs (FIG. 13C). Tunicamycin-induced expression of the 5× ATF6 reporter gene in IRE1α-null MEFs transfected with wild-type IRE1α was variable dependent on the tunicamycin concentration and duration of treatment (FIGS. 13B and 13C). Expression of the 50 kDa-processed form of ATF6 dramatically increased 5× ATF6 reporter activation in both cell types (FIG. 13D). Therefore, over-expression of the 50 kDa ATF6 bypassed the IRE1α requirement for 5× ATF6 reporter activation. Since ER stress-induction of the 5× ATF6 reporter was completely defective in IRE1α-null MEFs, but could be complemented by over-expression of 50 kDa processed form of ATF6, it was possible that IRE1α was required for ATF6 processing and/or function. Therefore, we studied the requirement for IRE1α in ATF6 cleavage and function.

### IRE1α is not Required for ATF6 Cleavage, Nuclear Translocation, or Transcriptional Activation.

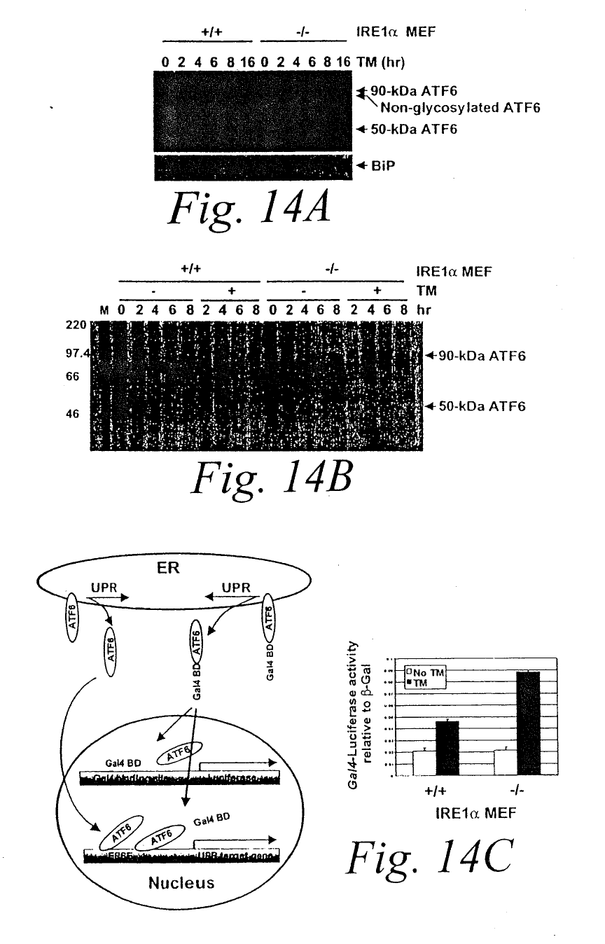

Initial studies demonstrated that IRE1α over-expression in COS-1 cells did not generate the processed form of ATF6 (data not shown). To further analyze the requirement for IRE1α in ATF6 function, ATF6 cleavage by Western blot and radiolabel pulse-chase experiments were examined. Cells were treated with tunicamycin for increasing amounts of time and ATF6 was monitored by Western blot analysis. The 50 kDa processed form of ATF6 was generated at the same rate in both wild-type and IRE1α-null MEFs and accumulated up to 8 hours (FIG. 14A, top). BiP protein levels also increased with similar kinetics in the wild-type and IRE1α-null MEFs (FIG. 14A, bottom). To more closely monitor the kinetics of 90 kDa ATF6 cleavage and stability, pulse-labeling with [³⁵S]-methionine and [³⁵S]-cysteine was performed with a chase in the presence or absence of tunicamycin. The labeled ATF6 proteins were immunoprecipitated with anti-ATF6 antibody and subjected to SDS-PAGE and autoradiography (FIG. 14B). The 50 kDa processed form of ATF6 was detected in both wild-type and IRE1α-null MEFs after 2 hours tunicamycin treatment. No significant difference in the cleavage and/or stability of ATF6 was detected between wild-type and IRE1α-null MEFs (FIG. 14B). Interestingly, both the intact and processed forms of ATF6 displayed a short half-life of approximately 2 hours.

To test whether ATF6 nuclear translocation and activation require IRE1α, a GAL4 transactivation assay was used. The GAL4 DNA binding domain was fused to the amino-terminus of full-length ATF6. This expression vector was transfected into wild-type and IRE1α-null MEFs with a luciferase reporter construct under transcriptional control of five GAL4 DNA binding sites. Under these conditions, the expression of luciferase is dependent on binding of the Gal4-ATF6 fusion protein liberated from the ER membrane (FIG. 14C, diagram). After cotransfection the cells were treated with tunicamycin. Tunicamycin induced luciferase expression in both wild-type and mutant MEFs to a similar degree, suggesting that cleavage, nuclear translocation and transcriptional activation of ATF6 are independent of IRE1α function (FIG. 14C). Therefore, by all these analyses, ATF6 processing and function were not defective in the IRE1α-null MEFs. These results led us to study whether another factor is defective in the IRE1α-null MEFs that is required for transcriptional activation of the 5x ATF6 reporter.

5× ATF6 Reporter Induction Requires IRE1α-Dependent Splicing of Xbp1 mRNA

XBP1 (X-box binding protein) is a bZIP transcription factor of the CREB/ATF protein family that binds to an identical sequence motif as ATF6 (Clauss et al., 1996) (FIG. 15A). Indeed, XBP1 was also isolated as an ERSE-binding factor in the same yeast one-hybrid screen used to identify ATF6 (Haze et al., 1999). During the course of our studies, it was discovered that two protein products are derived from the human Xbp1 mRNA, where the larger product is translated from a spliced form of Xbp1 mRNA that is generated upon ER stress (Yoshida et al, 2001b). Therefore, the sequence information was used to clone the full-length cDNA for murine Xbp1. The murine Xbp1 gene structure is very similar to the human Xbp1 having conserved two open reading frames, an intron, and a bZIP domain in the amino terminus (FIGS. 15B and 15C). The translation products from the 1st and 2nd open reading frames (ORFs) consist of 267 and 222 amino acids in the mouse and 261 and 212 amino acids in the human, respectively. Splicing of the intron would generate a frame-shift and a fusion of the 1st ORF to the 2nd ORF, to yield a larger protein product of 371 and 376 amino acids in the mouse and human, respectively. Only one base differs between the human and murine 26 base-intron. RT-PCR analysis of RNA isolated from tunicamycin-treated wild-type and IRE1α-null MEFs using PCR primers designed to amplify the region encompassing the overlap between ORF1 and ORF2 demonstrated that Xbp1 mRNA splicing is induced by ER stress and requires IRE1α (FIG. 15D). DNA sequence analysis confirmed the removal of 26 nucleotides from the shorter RT-PCR product. The 425-nt fragment from spliced Xbp1 mRNA was detected in wild-type MEFs after tunicamycin treatment. In contrast, this spliced form of Xbp1 mRNA was not detected in IRE1α-null MEFs before or after tunicamycin treatment. Western blot analysis using an antibody that reacts with only the longer XBP1 product derived from the spliced Xbp1 mRNA demonstrated a 55 kDa heterogenous-sized species that appeared with time after tunicamycin treatment in wild-type MEFs (FIG. 15E). This polypeptide was not detected before tunicamycin treatment. Although a small amount of the spliced Xbp1 mRNA was detected by RT-PCR prior to tunicamycin treatment, this analysis was not quantitative. Therefore, the presence of the spliced mRNA was not thought to be correlated with protein expression. This polypeptide was not detected in the IRE1α-null MEFs (FIG. 15E). Without being limited by theory, it is believed that this 55 kDa protein is translated from Xbp1 mRNA that is spliced in an IRE1α-dependent reaction. As expected from the presence of ERSE in the Xbp1 promoter and correct ATF6 processing in IRE1α-null MEFs, Xbp1 mRNA was induced with tunicamycin treatment in IRE1α-null MEFs (FIG. 15F).

If the defect in Xbp1 mRNA splicing was responsible for the defect in 5× ATF6 reporter induction in the IRE1α-null MEFs, then expression of the spliced form of Xbp1 mRNA, but not the unspliced form, should complement the 5× ATF6 reporter defect in the IRE1α-null MEFs. XBP1-ORF1 alone, XBP1-u (unspliced form of Xbp1) and XBP1-s (spliced form of Xbp1) were inserted behind the CMV promoter to direct their expression in transiently transfected COS-1 cells. Western blot analysis with antibody reactive to the amino-terminus of XBP1 detected a polypeptide of approximately 35 kDa in COS-1 cells transfected with the XBP1-ORF1 expression vector (FIG. 15G, lane 3). The 35 kDa polypeptide decreased upon tunicamycin treatment (lane 4), likely a consequence of decreased mRNA encoding the 35 kDa polypeptide due to splicing of Xbp1 mRNA. In addition, a 48 kDa species (asterisk) was induced upon tunicamycin treatment. The 48 kDa species may represent a product(s) from an aberrantly spliced mRNA(s) that utilizes the 5′ splice site junction in Xbp1 and a downstream cryptic 3′ splice site. Similar analysis of XBP1-u transfected cells detected the 35 kDa polypeptide in addition to a heterogeneous 55 kDa species representing XBP1-s. In contrast, cells transfected with XBP1-s produced only the latter 55 kDa species and its expression level did not change with tunicamycin treatment, likely because the CMV promoter is not induced by the UPR. These results demonstrate that each of the expression plasmids directs the expression of the expected polypeptide.

The effect of these expression vectors was then measured when cotransfected with the 5x ATF6 luciferase reporter gene into wild-type and IRE1α-null MEFs. Expression of either XBP1-ORF1 or intact unspliced XBP1-u slightly increased both the basal and tunicamycin-induced expression from the 5× ATF6 luciferase reporter gene in wild-type MEFs. In contrast, expression of XBP1-s greatly increased 5× ATF6 reporter gene expression in the wild-type MEFs, even in the absence of ER stress (FIG. 15H). Qualitatively similar results were obtained from cotransfection experiments in COS-1 cells (data not shown). Strikingly, only XBP1-s, complemented the 5× ATF6 reporter expression in the IRE1α-null MEFs. These results demonstrate that expression of the spliced form of Xbp1 mRNA is necessary and sufficient to activate the 5× ATF6 reporter gene in the IRE1α-null MEFs.

XBP1 mRNA is a Substrate of RNase Activity of IRE1α in vitro

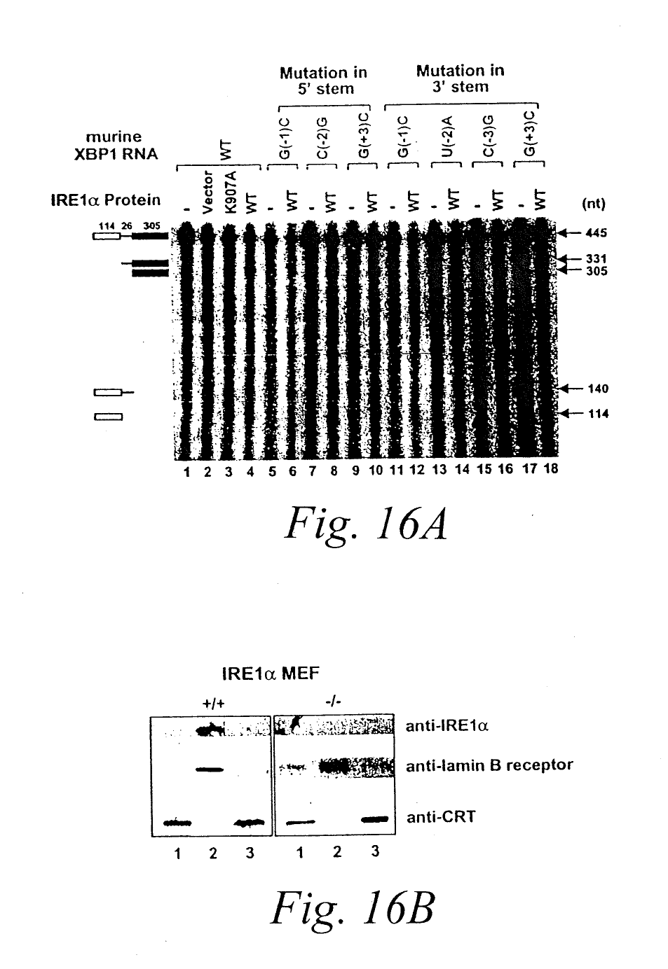

The predicted RNA structure of the Xbp1 intron shows stem-loop hairpins with 7-membered rings at both the 5′ and 3′ splice site junctions as observed in yeast HAC1 mRNA (FIG. 15C). Site-directed mutagenesis studies identified 3 residues (−1G, -3C, +3G) that are critical for cleavage of yeast HAC1 mRNA by IRE1p (Kawahara et al., 1998; Gonzalez et al., 1999). These bases are conserved in the 5′ and 3′ loops of Xbp1 mRNA (boxed in FIG. 15C). A test was preformed to determine whether Xbp1 mRNA is a direct substrate of the endoribonuclease activity of IRE1α in vitro and whether these conserved residues are required. Wild-type and mutant Xbp1 RNA substrates were transcribed in vitro and incubated with human IRE1α protein expressed in transfected COS-1 cells. Wild-type substrate was cleaved at both 5′ and 3′ splice site junctions (FIG. 16A, lane 4). Cleavage of the RNA at the 5′ or 3′ splice site was prevented by mutation of the conserved residues within the 5′ loop (−1G and +3G, lanes 6 and 10) or within the 3′ loop (−1G, −3C and +3G, lanes 12, 16 and 18), respectively. Mutation of the conserved residues in 5′ loop did not prevent cleavage of 3′ splice site and vice versa for mutations in the 3′ loop. In contrast, mutation of the nonconserved residue within the 5′ loop (−2C) or the 3′ loop (−2U) did not affect the cleavage of Xbp1 RNA by IRE1α (FIG. 16A, lanes 8 and 14). Taken together, these results support that both 5′ and 3′ splice site junctions in Xbp1 RNA are cleaved by IRE1α upon ER stress to eventually generate a spliced product that encodes a larger translated protein displaying greater transactivation potential.

### IRE1α Localizes to the Inner Nuclear Envelope

Previous studies suggest that the IRE1-mediated HAC1 mRNA splicing reaction may occur within the cytoplasm or the nucleus (Chapman and Walter, 1997; Ruegsegger et al., 2001). Cell fractionation was performed to localize IRE1a. Nuclei were isolated and their outer membranes were stripped as described in Materials and Methods. Western blot analysis of lamin B receptor demonstrated enrichment in the Triton X-100-insoluble fractions containing nuclei with the inner nuclear membrane (FIG. 16B). Lamin B was absent from the microsomal fraction containing the outer nuclear envelope. In contrast, calreticulin, a lumenal ER protein, was associated with the microsomal fraction. These results support that the nuclear and microsomal fractions isolated do not have significant contamination. Interestingly, IRE 1 a was greatly enriched in the nuclear pellet that was stripped of outer nuclear membranes. Importantly, the immunoreactivity was not detected in fractions isolated from IRE1α-null MEFs. These results support that the majority of IRE1α is localized to the inner nuclear envelope.

### IRE1α-Mediated UPR Transcriptional Induction Requires ATF6 Cleavage

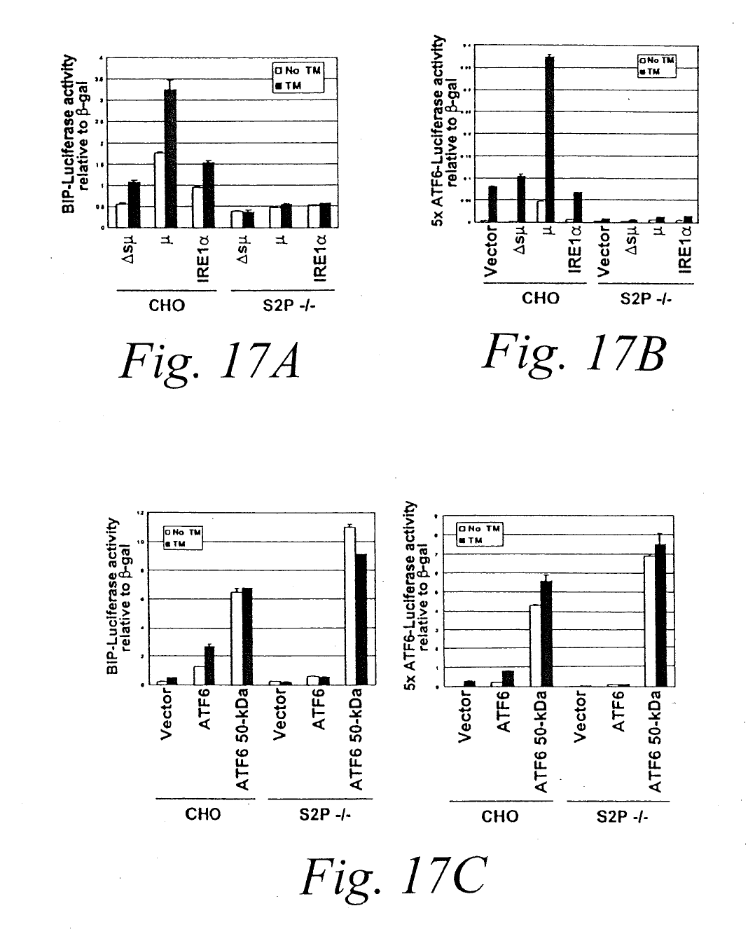

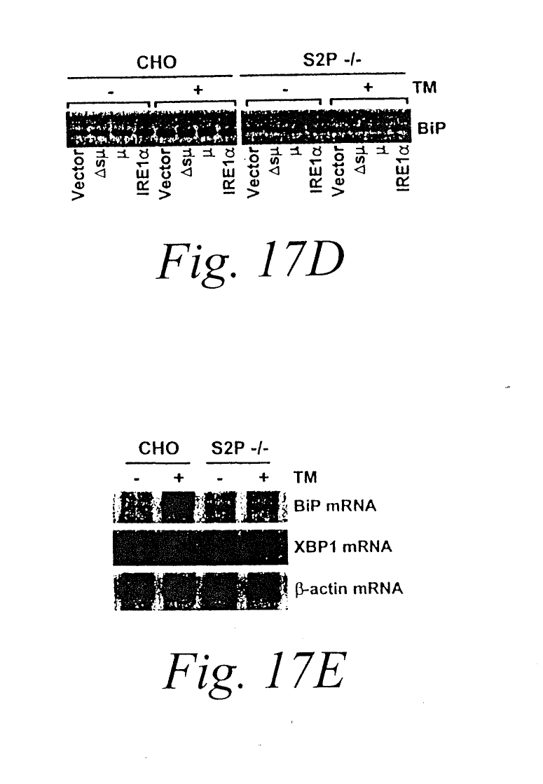

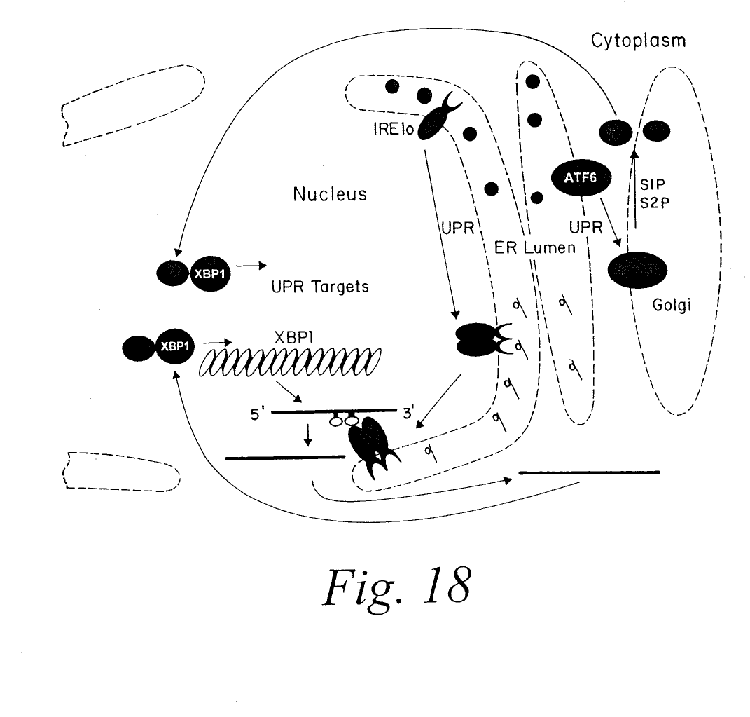

Site-1 protease (S1P) and site-2 protease (S2P) are implicated in the cleavage of ATF6 to generate the 50 kDa cytosolic fragment upon ER stress. Indeed, ATF6 cleavage was not detected in S2P-deficient CHO cells upon activation of the UPR (Ye et al., 2000). To test the requirement for ATF6 cleavage in IRE1α-mediated UPR transcriptional induction, we studied IRE1α over-expression in S2P-deficient CHO cells. Over-expression constitutively activates IRE1α by promoting dimer/oligomer formation and trans-autophosphorylation. An IRE1α expression vector was introduced into S2P-deficient CHO cells with a BiP promoter reporter plasmid or the 5× ATF6 reporter plasmid. IRE1α transfection in wild-type CHO cells increased BiP-reporter expression by 70% compared to cells transfected with immunoglobulin μ heavy chain deleted of the signal peptide (Asp.) (Wood et al., 1990) (FIG. 17A). In contrast, IRE1α transfection increased BiP reporter expression by 38% in S2P-deficient CHO cells. IRE1α over-expression reproducibly increased BiP reporter expression to a lower level in S2P-deficient CHO cells, suggesting that maximal IRE1α-mediated transcriptional induction requires S2P-dependent cleavage of ATF6. BiP expression was further increased by tunicamycin treatment in wild-type cells, but not in S2P-deficient CHO cells (FIG. 17A). Similarly, over-expression of immunoglobulin μ heavy chain, a known inducer of the UPR (Wood et al., 1990), increased BiP-reporter expression 208% in wild-type cells and only 23% in S2P-deficient CHO cells. In addition, over-expression of either IRE1α or immunoglobulin p heavy chain was not able to activate the 5× ATF6 reporter expression plasmid in S2P-deficient CHO cells, even in the presence of tunicamycin treatment (FIG. 17B). Northern and Western blot analysis of BiP in wild-type and S2P-deficient CHO cells revealed that S2P-dependent ATF6 processing is required for BiP induction upon tunicamycin-induced ER stress (FIGS. 17D and 17E). Indeed, over-expression of the 50 kDa processed form of ATF6, but not the full-length ATF6, rescued the UPR defect in S2P-deficient CHO cells (monitored by BiP-reporter or 5× ATF6 reporter expression) (FIG. 17C). BiP expression was not noticeably changed by over-expression of IRE1α or immunoglobulin p heavy chain even in wild-type CHO cells, probably because of the low transfection efficiency (FIG. 17D). These results support that ATF6 cleavage is required for induction of both IRE1α-dependent and ER stress-activated target genes. Finally, Xbp1 mRNA was induced in S2P-deficient CHO cells by tunicamycin treatment (FIG. 17E) suggesting that Xbp1 mRNA expression is regulated by IRE1α-dependent Xbp1 mRNA splicing, in addition to ATF6 cleavage (FIG. 18).

The references cited in Example 2 may be found in Lee, K. et al. Genes and Development 16: 452-466 (2002), expressly incorporated by reference herein.

### Example 3

### In Vivo IRE1 Activation Assay

In vivo activation of IRE1 can be monitored directly by phosphorylation of IRE1 or indirectly by identifying splicing of XBP1 mRNA. By western blot analysis, it is possible to distinguish unphosphorylated IRE1 from phosphorylated IRE1 due to the slower migration of the latter on reducing SDS-PAGE. Because the endogenous level of IRE1 expression is very low, IRE1 protein was detected by immunoprecipitation using an anti-IRE1 antibody and western blot analysis using the same antibody.

### A. Materials and Methods

### Transient Transfections

COS-1 monkey cells were transfected by either diethylaminoethyl (DEAE)-dextran (Kaufman, 1997) or Calcium Phosphate-BES methods (Ausubel et al, 1999). Chinese hamster ovary (CHO) cells were transfected by either lipofectAMINE PLUS (Life Technology) or FuGENE6 (Roche). Murine embryonic fibroblasts (MEFs) were transfected by either FuGENE6 (Roche) or Effectine (QIAGEN). IRE1 activation was monitored by immunoprecipitation and western analysis.

### RT-PCR and Reported Gene Expression

XBP1 splicing was also monitored by RT-PCR analysis of RNAs using primers designed to amplify the region encompassing the overlap between open reading frame 1 (ORF1) and ORF2 within XBP1 mRNA (see Lee et al., Genes and Dev. 2002). XBP1 splicing may also be monitored by assessing the expression of a reporter gene that is regulated by splicing of the XBP1 intron. The coding region of EGFP, Luciferase or β-galactosidase was fused to the mouse XBP1 ORF1 downstream of the XBP1 intron. Transcription of the construct is under control of the constitutively expressed CMV promoter. Therefore, expression of the XBP1-EGFP, XBP1-Luciferase or XBP1-β-galactosidase fusion protein is regulated by splicing of the XBP1 intron.

### B. Results

### Isolation of Nucleotide Sequence

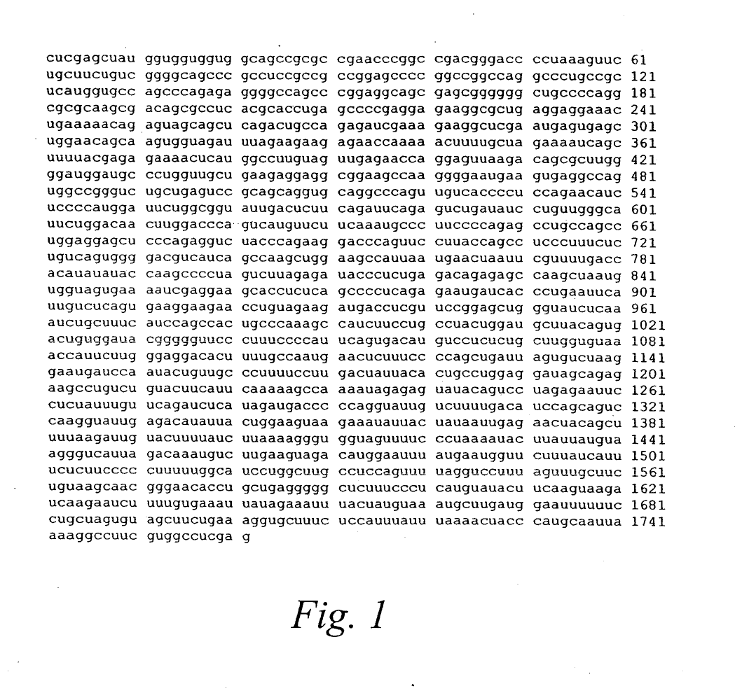

The human XBP1 spliced cDNA to mRNA sequence is shown in FIG. 1 and is set forth as SEQ ID NO:1. The protein encoded by this nucleic acid comprises about 376 amino acids and has the amino acid sequence shown in FIG. 2 and set forth as SEQ ID NO:2. The coding region (open reading frame) of the human spliced cDNA sequence is shown in FIG. 3 and is set forth as SEQ ID NO:3. The coding region (open reading frame) of the human unspliced cDNA sequence is shown in FIG. 4 and is set forth as SEQ ID NO:4. The human XBP1 spliced cDNA sequence was deposited with the Gen Bank Database and assigned Accession No. AB076384. The human XBP1 unspliced cDNA sequence was also deposited with the Gen Bank Database and assigned Accession No. AB076383.

Transient Transfection of XBP1-EGFP into COS-1 Cells

At 48 hours after transfection, cells were treated with 10 ug/ml tunicamycin to inhibit N-linked glycosylation. Where control cells demonstrated no fluorescence, cells transfected with the XBP1-EGFP construct displayed bright fluoresence after 8 and 16 hours. Upon expression of the XBP1-EGFP construct in murine embryonic fibroblast cells that are deleted in both IRE1α alleles, no fluorescence was detected, in contrast to control cells where intense fluorescsence was observed upon treatment with tunicamycin. Therefore, this construct is instrumental in monitoring IRE1 activation.

### Stable Cell Lines

To create stable cell lines expressing the XBP1-reporter fusion transcripts, various cell lines are transfected with pcDNA3-XBP1-EGFP, pcDNA3-XBP1-Luciferase or pcDNA3-XBP1-β-galactosidase and selected with neomycin (Geneticin) in complete media. Cells are allowed to double twice under nonselective conditions and ten times under selection conditions, respectively, before individual colonies are picked and expanded into cell lines.

### Transgenic Animals

In order to generate mice transgenic for XBP1-EGFP or XBP1-β-galactosidase transgenes, linear DNA fragments were microinjected into fertilized mouse eggs. Transgenic founders are identified by PCR. Southern analysis is performed to determine the copy number, integration site number, and transgene integrity in the transgenic founder mice prior to breeding.

### Over-Expression of Spliced XBP1 Expands the Volume of the Endoplasmic Reticulum

CHO cells were transfected with an expression vector that contains the spliced form of XBP1. Cells were transfected with a selectable plasmid that directs puromycin resistance. After 48 hours, cells were treated with 10 mg/ml purimycin for 16 hours to kill non-transfected cells. Then cells were prepared for electron microscopy. An electron microscope image was taken of cells transfected with IRE1α alone, spliced XBP1 alone, the processed form of ATF6 alone, and all three expression plasmids together. Only expression of the spliced form of XBP1 activates expansion of the endoplasmic reticulum compartment. Cells that express spliced XBP1 in the absence of ER stress may provide a means to more efficiently express proteins that transit the secretory pathway.

### Equivalents

Those skilled in the art will recognize, or be able to ascertain using no more than routine experimentation, many equivalents to the specific embodiments of the invention described herein. Such equivalents are intended to be encompassed by the following claims.

1-52. (canceled)

53. A pharmaceutical composition comprising an isolated nucleic acid molecule selected from the group consisting of:
(a) a nucleic acid molecule comprising the nucleotide sequence set forth in SEQ ID NO:1; and
(b) a nucleic acid molecule comprising the nucleotide sequence set forth in SEQ ID NO:3
in combination with; a pharmaceutically acceptable excipient.

54. A pharmaceutical composition comprising an isolated nucleic acid molecule which encodes a polypeptide comprising the amino acid sequence set forth in SEQ ID NO:2 in combination with; a pharmaceutically acceptable excipient.

55. A pharmaceutical composition comprising an isolated nucleic acid molecule that encodes a polypeptide having X-box binding protein 1 activity selected from the group consisting of:
a. a nucleic acid molecule comprising a nucleotide sequence which is at least 98% identical to the nucleotide sequence of SEQ ID NO:1 or 3; and
b. a nucleic acid molecule which encodes a polypeptide comprising an amino acid sequence at least 98% identical to the amino acid sequence of SEQ ID NO:2 in combination with; a pharmaceutically acceptable excipient.

56. (canceled)

57. A pharmaceutical composition comprising an isolated nucleic acid molecule comprising the nucleic acid molecule of claim 1 and a nucleic acid molecule encoding a heterologous polypeptide in combination with a pharmaceutically acceptable excipient.

58. A pharmaceutical composition comprising an expression vector comprising a nucleic acid molecule selected from the group consisting of:
a. a nucleic acid molecule comprising the nucleotide sequence set forth in SEQ ID NO:1; and
b. a nucleic acid molecule comprising the nucleotide sequence set forth in SEQ ID NO:3 in combination with; a pharmaceutically acceptable excipient.

59. A pharmaceutical composition comprising an isolated host cell transfected with the expression vector of claim 58.

60. (canceled)

61. A kit comprising the pharmaceutical composition as in any one of the preceding claims.

62. A method of producing a pharmaceutical composition of a polypeptide comprising culturing the host cell of claim 59 in an appropriate culture medium to, thereby produce the polypeptide.


SEQUENCE LISTING (45 sequences)

SEQ ID NO: 1 - DNA, 1761 bp, Homo sapiens
cucgagcuaugguggugguggcagccgcgccgaacccggccgacgggaccccuaaaguucugcuucugucggggcagcccgccuccgccgccggagccccggccggccaggcccugccgcucauggugccagcccagagaggggccagcccggaggcagcgagcggggggcugccccaggcgcgcaagcgacagcgccucacgcaccugagccccgaggagaaggcgcugaggaggaaacugaaaaacagaguagcagcucagacugccagagaucgaaagaaggcucgaaugagugagcuggaacagcaagugguagauuuagaagaagagaaccaaaaacuuuugcuagaaaaucagcuuuuacgagagaaaacucauggccuuguaguugagaaccaggaguuaagacagcgcuuggggauggaugcccugguugcugaagaggaggcggaagccaaggggaaugaagugaggccaguggccgggucugcugaguccgcagcaggugcaggcccaguugucaccccuccagaacaucuccccauggauucuggcgguauugacucuucagauucagagucugauauccuguugggcauucuggacaacuuggacccagucauguucuucaaaugcccuuccccagagccugccagccuggaggagcucccagaggucuacccagaaggacccaguuccuuaccagccucccuuucucugucaguggggacgucaucagccaagcuggaagccauuaaugaacuaauucguuuugaccacauauauaccaagccccuagucuuagagauacccucugagacagagagccaagcuaaugugguagugaaaaucgaggaagcaccucucagccccucagagaaugaucacccugaauucauugucucagugaaggaagaaccuguagaagaugaccucguuccggagcuggguaucucaaaucugcuuucauccagccacugcccaaagccaucuuccugccuacuggaugcuuacagugacuguggauacggggguucccuuuccccauucagugacauguccucucugcuugguguaaaccauucuugggaggacacuuuugccaaugaacucuuuccccagcugauuagugucuaaggaaugauccaauacuguugcccuuuuccuugacuauuacacugccuggaggauagcagagaagccugucuguacuucauucaaaaagccaaaauagagaguauacaguccuagagaauuccucuauuuguucagaucucauagaugacccccagguauugucuuuugacauccagcaguccaagguauugagacauauuacuggaaguaagaaauauuacuauaauugagaacuacagcuuuuaagauuguacuuuuaucuuaaaagggugguaguuuucccuaaaauacuuauuauguaagggucauuagacaaaugucuugaaguagacauggaauuuaugaaugguucuuuaucauuucucuucccccuuuuuggcauccuggcuugccuccaguuuuagguccuuuaguuugcuucuguaagcaacgggaacaccugcugagggggcucuuucccucauguauacuucaaguaagaucaagaaucuuuugugaaauuauagaaauuuacuauguaaaugcuugauggaauuuuuuccugcuaguguagcuucugaaaggugcuuucuccauuuauuuaaaacuacccaugcaauuaaaaggccuucguggccucgag

SEQ ID NO: 2 - protein, 376 aa, Homo sapiens
MVVVAAAPNPADGTPKVLLLSGQPASAAGAPAGQALPLMVPAQRGASPEAASGGLPQARKRQRLTHLSPEEKALRRKLKNRVAAQTARDRKKARMSELEQQVVDLEEENQKLLLENQLLREKTHGLVVENQELRQRLGMDALVAEEEAEAKGNEVRPVAGSAESAAGAGPVVTPPEHLPMDSGGIDSSDSESDILLGILDNLDPVMFFKCPSPEPASLEELPEVYPEGPSSLPASLSLSVGTSSAKLEAINELIRFDHIYTKPLVLEIPSETESQANVVVKIEEAPLSPSENDHPEFIVSVKEEPVEDDLVPELGISNLLSSSHCPKPSSCLLDAYSDCGYGGSLSPFSDMSSLLGVNHSWEDTFANELFPQLISV

SEQ ID NO: 3 - DNA, 1761 bp, Homo sapiens
ctcgagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggccttcgtggcctcgag

SEQ ID NO: 4 - DNA, 1787 bp, Homo sapiens
ctcgagctatggtggtggtggcagccgcgccgaacccggccgacgggacccctaaagttctgcttctgtcggggcagcccgcctccgccgccggagccccggccggccaggccctgccgctcatggtgccagcccagagaggggccagcccggaggcagcgagcggggggctgccccaggcgcgcaagcgacagcgcctcacgcacctgagccccgaggagaaggcgctgaggaggaaactgaaaaacagagtagcagctcagactgccagagatcgaaagaaggctcgaatgagtgagctggaacagcaagtggtagatttagaagaagagaaccaaaaacttttgctagaaaatcagcttttacgagagaaaactcatggccttgtagttgagaaccaggagttaagacagcgcttggggatggatgccctggttgctgaagaggaggcggaagccaaggggaatgaagtgaggccagtggccgggtctgctgagtccgcagcactcagactacgtgcacctctgcagcaggtgcaggcccagttgtcacccctccagaacatctccccatggattctggcggtattgactcttcagattcagagtctgatatcctgttgggcattctggacaacttggacccagtcatgttcttcaaatgcccttccccagagcctgccagcctggaggagctcccagaggtctacccagaaggacccagttccttaccagcctccctttctctgtcagtggggacgtcatcagccaagctggaagccattaatgaactaattcgttttgaccacatatataccaagcccctagtcttagagataccctctgagacagagagccaagctaatgtggtagtgaaaatcgaggaagcacctctcagcccctcagagaatgatcaccctgaattcattgtctcagtgaaggaagaacctgtagaagatgacctcgttccggagctgggtatctcaaatctgctttcatccagccactgcccaaagccatcttcctgcctactggatgcttacagtgactgtggatacgggggttccctttccccattcagtgacatgtcctctctgcttggtgtaaaccattcttgggaggacacttttgccaatgaactctttccccagctgattagtgtctaaggaatgatccaatactgttgcccttttccttgactattacactgcctggaggatagcagagaagcctgtctgtacttcattcaaaaagccaaaatagagagtatacagtcctagagaattcctctatttgttcagatctcatagatgacccccaggtattgtcttttgacatccagcagtccaaggtattgagacatattactggaagtaagaaatattactataattgagaactacagcttttaagattgtacttttatcttaaaagggtggtagttttccctaaaatacttattatgtaagggtcattagacaaatgtcttgaagtagacatggaatttatgaatggttctttatcatttctcttccccctttttggcatcctggcttgcctccagttttaggtcctttagtttgcttctgtaagcaacgggaacacctgctgagggggctctttccctcatgtatacttcaagtaagatcaagaatcttttgtgaaattatagaaatttactatgtaaatgcttgatggaattttttcctgctagtgtagcttctgaaaggtgctttctccatttatttaaaactacccatgcaattaaaaggccttcgtggcctcgag

SEQ ID NO: 5 - DNA, 20 bp, Artificial Sequence
  primer
ccttgtggttgagaaccagg

SEQ ID NO: 6 - DNA, 19 bp, Artificial Sequence
  primer
ctagaggcttggtgtatac

SEQ ID NO: 7 - DNA, 23 bp, Artificial Sequence
  primer
cgaattcttagacactaatcagc

SEQ ID NO: 8 - DNA, 19 bp, Artificial Sequence
  primer
tctgctgagtccccagcac

SEQ ID NO: 9 - DNA, 19 bp, Artificial Sequence
  primer
gtgctggggactcagcaga

SEQ ID NO: 10 - DNA, 19 bp, Artificial Sequence
  primer
tctgctgagtcggcagcac

SEQ ID NO: 11 - DNA, 19 bp, Artificial Sequence
  primer
gtgctgccgactcagcaga

SEQ ID NO: 12 - DNA, 20 bp, Artificial Sequence
  primer
gtccgcaccactcagactat

SEQ ID NO: 13 - DNA, 20 bp, Artificial Sequence
  primer
atagtctgagtggtgcggac

SEQ ID NO: 14 - DNA, 19 bp, Artificial Sequence
  primer
atgtgcacctctccagcag

SEQ ID NO: 15 - DNA, 19 bp, Artificial Sequence
  primer
ctgctggagaggtgcacat

SEQ ID NO: 16 - DNA, 19 bp, Artificial Sequence
  primer
atgtgcacctcagcagcag

SEQ ID NO: 17 - DNA, 19 bp, Artificial Sequence
  primer
ctgctgctgaggtgcacat

SEQ ID NO: 18 - DNA, 19 bp, Artificial Sequence
  primer
atgtgcacctgtgcagcag

SEQ ID NO: 19 - DNA, 19 bp, Artificial Sequence
  primer
ctgctgcacaggtgcacat

SEQ ID NO: 20 - DNA, 19 bp, Artificial Sequence
  primer
ctctgcaccaggtgcaggc

SEQ ID NO: 21 - DNA, 19 bp, Artificial Sequence
  primer
gcctgcacctggtgcagag

SEQ ID NO: 22 - DNA, 19 bp, Caenorhabditis elegans
ccaatcggcgagcgccgcg

SEQ ID NO: 23 - DNA, 19 bp, Caenorhabditis elegans
cctatcgtcctaggccacg

SEQ ID NO: 24 - DNA, 19 bp, Caenorhabditis elegans
cgaatgctcaaggtcgact

SEQ ID NO: 25 - DNA, 16 bp, Caenorhabditis elegans
attggccccgcccaca

SEQ ID NO: 26 - DNA, 16 bp, Caenorhabditis elegans
attggccccgcccaca

SEQ ID NO: 27 - DNA, 26 bp, Caenorhabditis elegans
gttttcagtttgacgtttagaggttg

SEQ ID NO: 28 - DNA, 26 bp, Caenorhabditis elegans
acctagtaactgacgtgtcccacgga

SEQ ID NO: 29 - DNA, 26 bp, Caenorhabditis elegans
ttcgctgtgctgacgttgccttgtcc

SEQ ID NO: 30 - DNA, 26 bp, Caenorhabditis elegans
ttatcggagatgacgtgtattagtgc

SEQ ID NO: 31 - DNA, 48 bp, Caenorhabditis elegans
gcctttgaatcagcagcattcattaatgagcctcagcagtgggaacag

SEQ ID NO: 32 - DNA, 25 bp, Caenorhabditis elegans
gcctttgaatcagcagtgggaacag

SEQ ID NO: 33 - RNA, 50 nt, Caenorhabditis elegans
gccuuugaaucagcagcauucauuaaugagccugagcagugggaacaggc

SEQ ID NO: 34 - RNA, 21 nt, Saccharomyces cerevisiae
cguaauccagccgugauuacg

SEQ ID NO: 35 - RNA, 17 nt, Saccharomyces cerevisiae
acuguccgaagcgcagu

SEQ ID NO: 36 - DNA, 5 bp, Mus musculus
ccacg

SEQ ID NO: 37 - DNA, 27 bp, Mus musculus
ctcgagacaggtgctgacgtggcattc

SEQ ID NO: 38 - DNA, 15 bp, Mus musculus
  Misc_feature (11)..(13): G or C or A or T
gatgacgtggnnntt

SEQ ID NO: 39 - DNA, 15 bp, Mus musculus
  Misc_feature (11)..(13): G or C or A or T
gatgacgtgtnnntt

SEQ ID NO: 40 - DNA, 15 bp, Mus musculus
  Misc_feature (11)..(13): G or C or A or T
gatgacgtgtnnnat

SEQ ID NO: 41 - DNA, 15 bp, Mus musculus
  Misc_feature (11)..(13): G or C or A or T
gatgacgtggnnnat

SEQ ID NO: 42 - DNA, 13 bp, Mus musculus
  Misc_feature (10)..(12): G or C or A or T
gtgacgtggnnnt

SEQ ID NO: 43 - DNA, 8 bp, Mus musculus
tgacgtca

SEQ ID NO: 44 - DNA, 19 bp, Mus musculus
  Misc_feature (06)..(14): G or C or A or T
ccaatnnnnnnnnnccacg

SEQ ID NO: 45 - RNA, 58 nt, Mus musculus
ggucugcugaguccgcagcacucagacuaugugcaccucugcagcaggugcggcccag